(19) United States
(12) Patent Application Publication
(10) Pub. No.: US 2025/0302612 A1
(43) Pub. Date: Oct. 2, 2025

(54) COMMISSURE ASSEMBLY FOR PROSTHETIC HEART VALVES

(71) Applicant: EDWARDS LIFESCIENCES CORPORATION, Irvine, CA (US)

(72) Inventors: Danny M. Garmahi, Hadera (IL); Roy Shitrit, Zichron Yaakov (IL); Nikolai Gurovich, Hadera (IL); Tamir S. Levi, Zikhron Yaakov (IL); Brendan Michael Dalbow, Oceanside, CA (US); Elena Sherman, Pardes Hana (IL); Svetlana Morduchovitch, Be'er Ya'akov (IL)

(73) Assignee: EDWARDS LIFESCIENCES CORPORATION, Irvine, CA (US)

(21) Appl. No.: 19/232,007

(22) Filed: Jun. 9, 2025

Related U.S. Application Data
(63) Continuation of application No. PCT/US2023/083334, filed on Dec. 11, 2023.
(60) Provisional application No. 63/432,602, filed on Dec. 14, 2022.
(60) Provisional application No. 63/602,922, filed on Nov. 27, 2023.

(51) Int. Cl. A61F 2/24

(52) U.S. Cl. CPC A61F 2/2418; A61F 2/2415; A61F 2220/0075

(57) ABSTRACT

A prosthetic heart valve includes a radially expandable frame and a valvular structure having a plurality of leaflets. The frame includes one or more commissure windows, with two window struts, to receive and support the leaflets. Each leaflet is attached to the frame by a commissure tab which extends through a commissure window. A reinforcing member is attached to the commissure tab of each leaflet by one or more suture lines. Adjacent commissure tabs and reinforcing members are attached to the window struts by being folded around them and secured with one or more securing loops. In some examples, the commissure tabs can be attached to the window struts without any load-bearing sutures extending through the leaflets.

### CROSS-REFERENCE TO RELATED APPLICATIONS

This application is a continuation of PCT Application No. PCT/US2023/083334, filed Dec. 11, 2023, which claims the benefit of U.S. Provisional Patent Application No. 63/432,602, filed Dec. 14, 2022, and U.S. Provisional Patent Application No. 63/602,922 filed Nov. 27, 2023, which are each incorporated by reference herein in their entireties.

### FIELD

The present disclosure relates to prosthetic heart valves, commissure assemblies for prosthetic heart valves, and methods for implanting the same.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

The prosthetic heart valve can include valvular structure (formed by two or more leaflets) for regulating blood flow in a single direction. The leaflets can form commissures between adjacent ones of the leaflets. The commissures can be attached to a frame of the prosthetic heart valve.

### SUMMARY

The foregoing and other objects, features, and advantages of the invention will become more apparent from the following detailed description, which proceeds with reference to the accompanying figures.

A prosthetic heart valve can comprise a frame and a valvular structure coupled to the frame. The frame of the prosthetic heart valve can comprise a commissure window defined by a first axially extending post and a second axially extending post. In addition to these components, a prosthetic heart valve can further comprise one or more of the components disclosed herein.

In some examples, prosthetic heart valve can comprise a commissure assembly having a commissure tab forming one or more right angle or substantially right-angle bends around one of the first or second axially extending posts.

In some examples, prosthetic heart valve can comprise a commissure assembly having a commissure tab forming two or more right angle or substantially right-angle bends around one of the first or second axially extending posts.

In some examples, prosthetic heart valve can comprise a commissure assembly having a reinforcing member secured to a leaflet by one or more suture lines, and one or more securing loops can extend between the one or more suture lines to secure the commissure assembly to the frame.

In some examples, a prosthetic heart valve can comprise a commissure assembly having a reinforcing member secured to a leaflet and including two or more suture lines, and one or more securing loops can extend between the two or more suture lines to secure the commissure assembly to the frame.

In some examples, prosthetic heart valve can comprise a commissure assembly having a reinforcing member secured to a leaflet and including three or more suture lines, and two or more securing loops can extend between the three or more suture lines to secure the commissure assembly to the frame.

In some examples, a reinforcing member can comprise one or more suture lines that extend through the reinforcing member and a commissure tab.

In some examples, a reinforcing member can comprise one or more suture lines that extend only through the reinforcing member and do not extend through a commissure tab.

In some examples, a suture line extending through the reinforcing member and a commissure tab can be attached to a wedge member via a connecting loop.

In some examples, a suture line extending only through the reinforcing member can be connected to another suture line extending only through a reinforcing member on an adjacent leaflet via a connecting loop.

In some examples, a reinforcing member can form one or more folded portions when wrapped over or around a commissure window strut.

In some examples, an end portion of a commissure tab inserted through a commissure window can be sandwiched between a first folded portion and a second folded portion of a reinforcing member.

In some examples, a folded portion of a reinforcing member on an exterior of a frame can be connected to another folded portion of a reinforcing member on an adjacent leaflet via a connecting loop.

In some examples, prosthetic heart valve can comprise a commissure assembly wherein no leaflet of the valvular structure is attached to itself or any other leaflet by a suture line extending through a leaflet.

In some examples, prosthetic heart valve can comprise a commissure assembly having a commissure tab that is parallel to two or more faces of the first axially extending post or the second axially extending post.

In some examples, prosthetic heart valve can comprise a commissure assembly having a commissure tab that is parallel to three or more faces of the first axially extending post or the second axially extending post.

In some examples, prosthetic heart valve can comprise a commissure assembly having a reinforcing member enclosing the first axially extending post or the second axially extending post.

Some examples concern a prosthetic heart valve comprising a radially expandable frame having a first end portion, a second end portion and a longitudinal axis extending between the first end portion and the second end portion, and a plurality of commissure windows. The prosthetic heart valve also comprises a valvular structure disposed within and attached to the radially expandable frame, the valvular structure comprising at least a first leaflet having a first commissure tab and a first reinforcing member attached to the first commissure tab and a second leaflet having a second commissure tab and a second reinforcing member attached to the second commissure tab. The first and second commissure tabs are positioned adjacent each other and extend through a first commissure window of the plurality of commissure windows, the first commissure window being defined by first and second axially extending posts. The first commissure tab forms two or more bends around the first axially extending post and the second commissure tab forms two or more bends around the second axially extending post.

Some examples concern a commissure assembly for a prosthetic heart valve. The prosthetic heart valve comprises a radially expandable frame with a commissure window having a first axially extending post and a second axially extending post. The commissure assembly comprises at least a first leaflet having a first commissure tab and a second leaflet having a second commissure tab, a first reinforcing member sutured to the first commissure tab and a second reinforcing member sutured to the second commissure tab, a first suture line and a second suture line extending through the first reinforcing member and the first leaflet, a third suture line extending through the first reinforcing member, a first securing loop extending between the second suture line and the third suture line, and a second securing loop extending between the first suture line and the third suture line. The first commissure tab and the second commissure tab extend through the commissure window to form a commissure. The first securing loop extends around the first axially extending post of the commissure window, and the second securing loop extends around an end portion of the first leaflet. The first commissure tab is folded to at least partially enclose the first axially extending post.

Some examples concern a prosthetic heart valve, comprising a radially expandable frame having a first end portion, a second end portion, and a longitudinal axis extending between the first end portion and the second end portion, and a plurality of commissure windows. The prosthetic heart valve also comprises a valvular structure disposed within and attached to the radially expandable frame, the valvular structure comprising at least a first leaflet having a first commissure tab and a first reinforcing member attached to the first commissure tab and a second leaflet having a second commissure tab and a second reinforcing member attached to the second commissure tab. The first and second commissure tabs are positioned adjacent each other and extend through a first commissure window of the plurality of commissure windows, the first commissure window being defined by first and second axially extending posts. The first reinforcing member is attached to the first commissure tab by a first suture line, and no suture line attaches any leaflet to itself or to another leaflet.

Some examples concern a prosthetic heart valve, comprising a radially expandable frame having a first end portion, a second end portion, and a longitudinal axis extending between the first end portion and the second end portion, and a plurality of commissure windows. The prosthetic heart valve also comprises a plurality of leaflets disposed within and attached to the radially expandable frame and forming a valvular structure configured to regulate a flow of blood through the radially expandable frame. A first leaflet of the plurality of leaflets comprises a first commissure tab and a first reinforcing member is attached to the first commissure tab. The first commissure tab extends through a first commissure window of the plurality of commissure windows defined by a first axially extending post and a second axially extending post. The first axially extending post has a rectangular cross section and the first commissure tab extends parallel to a first face, a second face, and a third face of the first axially extending post when secured.

Some examples concern a prosthetic heart valve, comprising a radially expandable frame having a first end portion, a second end portion, and a longitudinal axis extending between the first end portion and the second end portion, and a plurality of commissure windows. The prosthetic heart valve also comprises a plurality of leaflets disposed within and attached to the radially expandable frame and forming a valvular structure configured to regulate a flow of blood through the radially expandable frame. A plurality of reinforcing members are attached to the plurality of leaflets. A first leaflet of the plurality of leaflets comprises a first commissure tab and a first reinforcing member is attached to the first commissure tab. The first commissure tab extends through a first commissure window of the plurality of commissure windows defined by a first axially extending post and a second axially extending post. The first axially extending post has a rectangular cross section, and wherein the first reinforcing member encloses the first axially extending post.

Some examples concern a method of attaching a leaflet to a prosthetic heart valve, comprising attaching a reinforcing member to a leaflet with a first suture line and a second suture line to form a leaflet assembly, the second suture line being spaced apart from the first suture line along a commissure tab of the leaflet, the first suture line and the second suture line extending through the reinforcing member and the leaflet. The method also comprises attaching a third suture line to the reinforcing member, spaced apart from the second suture line along a length of the reinforcing member. The method also comprises arranging the leaflet assembly inside a radially expandable frame comprising a commissure window defined by a first axially extending window strut and a second axially extending window strut, such that an end portion of the leaflet assembly extends through the commissure window with the third suture line being positioned radially inwards of the commissure window and the first suture line and the second suture line being positioned radially outwards from the commissure window. The method also comprises folding a first end portion of the reinforcing member around the first axially extending window strut and folding the end portion of the leaflet at least partially around the first axially extending window strut and the reinforcing member.

Some examples concern a method of assembling a prosthetic heart valve, comprising assembling a first leaflet assembly by suturing a first reinforcing member to a first leaflet with first and second suture lines extending through the first leaflet and the first reinforcing member. The method also comprises forming a third suture line along the first reinforcing member and inserting a first commissure tab of the first leaflet assembly through a first commissure window of a frame of the prosthetic heart valve. The method also comprises forming a first securing loop that extends through the second and third suture lines and around a first axially extending window strut of the first commissure window and tightening the first securing loop to fold the first reinforcing member and the first commissure tab around the first axially extending window strut.

In some examples, a prosthetic heart valve can comprise one or more of the components recited in Examples 1-70 and 92-113 below.

In some examples, a method can comprise one or more of the steps recited in Examples 71-91 and 114-117 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of an example of a delivery apparatus configured to deliver and implant a radially expandable prosthetic heart valve at an implantation site.

FIG. 3 is a side elevation view of a prosthetic heart valve, according to another example.

FIG. 4 is a front view of a leaflet according to one example.

FIG. 5 is a front view of a portion of the leaflet of FIG. 4 with an attached reinforcing member according to one example.

FIG. 6A is a top plan view of the portion of the leaflet with the attached reinforcing member of FIG. 5, prior to assembly with a frame of the prosthetic heart valve.

FIGS. 6B-6E are top plan views of the leaflet and attached reinforcing member of FIG. 6A at various stages of assembly within one of the commissure windows of the frame.

FIG. 7 is a sectioned top plan view of a commissure window with attached commissure according to the exemplary stages of assembly illustrated in FIGS. 6A-6E.

FIG. 8 is a front view of a leaflet with attached reinforcing members according to another example.

FIG. 9 is a sectioned top plan view of a first stage of assembly of a commissure at a commissure window for leaflets with attached reinforcing members as shown in FIG. 8.

FIG. 10 is a sectioned top plan view of a first exemplary commissure formed at the commissure window after the first stage of assembly shown in FIG. 9.

FIG. 11 is a sectioned top plan view of a second exemplary commissure formed at the commissure window after the first stage of assembly shown in FIG. 9.

FIG. 12 is a front view of a leaflet with attached reinforcing members according to another example.

FIGS. 13-15 are sectioned top plan views at various stages of assembly within one of the commissure windows of the frame for leaflets with attached reinforcing members as shown in FIG. 12.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

As discussed above, leaflets of a valvular structure for a prosthetic heart valve can form commissures between adjacent ones of the leaflets, which can be attached to a frame of the prosthetic valve. In some examples, suturing of the leaflets to themselves and/or to adjacent leaflets can undesirably increase the bulk of the commissures (which can, for example, increase an overall crimp profile of the prosthetic valve and make transcatheter delivery of the radially compressed valve more difficult). In some examples, suturing of the leaflets to themselves and/or to adjacent leaflets can undesirably increase load-bearing on the leaflets during operative opening and closing cycles of the valvular structure (which can, for examples, result in wear and/or deformation of the leaflets). In some examples, suturing of the leaflets to themselves and/or to adjacent leaflets can undesirably increase complexity and/or assembly time for the prosthetic heart valve.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

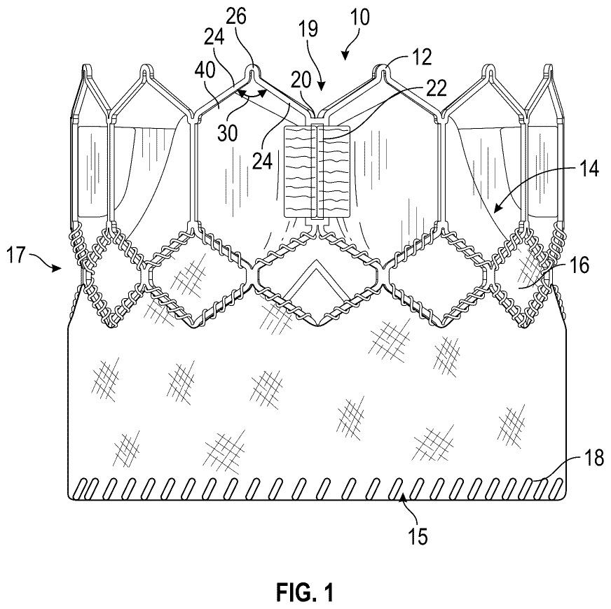

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

Described herein are examples of radially expandable and compressible prosthetic heart valves including an annular frame. In some examples, the frame of the prosthetic heart valve can include a plurality of rows of cells formed by interconnected struts of the frame. The plurality of rows of cells can be formed between an inflow end and an outflow end of the frame.

The prosthetic heart valve may further include a plurality of leaflets attached to the frame. In some examples, the leaflets can be attached to the frame via commissures formed by joining pairs of adjacent ends (for example, commissure tabs) of the leaflets. In some examples, one or more leaflets of the prosthetic heart valve can comprise a connecting suture line along a cusp edge portion of the leaflet. The connecting suture line can be attached to the leaflet prior to attachment of the leaflet to the frame. The connecting suture line can be further configured to enable direct attachment of the cusp edge portion of the leaflet to a portion of struts of the frame, without being attached to an inner skirt and without extending additional sutures through the cusp edge portion of the leaflets.

In some examples, the leaflets can include reinforcing members attached to commissure tabs extending from opposing sides of the leaflet. The reinforcing members can include one or more suture lines that extend through the reinforcing member and the commissure tab. In some examples, the reinforcing members can further include one or more suture lines that extend only through the reinforcing member and do not extend through the commissure tab.

Commissure tabs of adjacent leaflets can be paired for formation of a commissure therebetween. After passing the pair of commissure tabs (each having a reinforcing member attached thereto) through a commissure window formed by commissure window struts of the frame, the commissure tabs and the reinforcing members can be wrapped over one or more faces of the struts. In some examples, the commissure tabs can be wrapped over two or more faces of the struts, while the reinforcing members are wrapped around all four faces of the struts. In some examples the reinforcing members are disposed between the surfaces of the struts that the commissure tabs. A wedge member can be secured to the pair of commissure tabs on an outside of the frame by attaching the wedge member to suture lines that extends through the reinforcing member and the commissure tab via one or more securing loops. Additional securing loops can be stitched through or around sutures that extend only through the reinforcing members or folded portions of the reinforcing members, thereby forming a commissure that can prevent the commissure tabs from being pulled back through the commissure window and securing the leaflets to the frame.

Exemplary leaflets including reinforcing members, methods of assembly, and commissure configurations are illustrated in FIGS. 5-15.

### Examples of the Disclosed Technology

The prosthetic valve 10 of FIG. 1 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, Nitinol). When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frames disclosed herein (e.g., the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference.

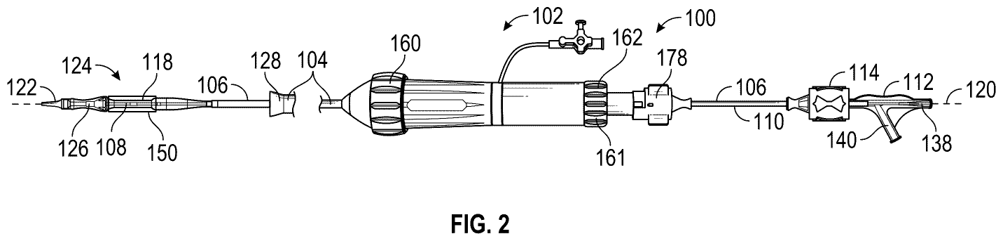

FIG. 2 shows a delivery apparatus 100, according to an example, that can be used to implant an expandable prosthetic heart valve (for example, the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (that is, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

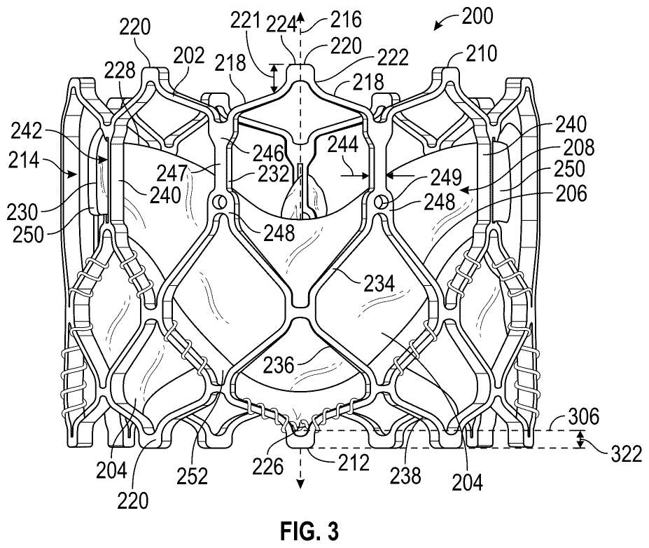

FIG. 3 shows an example of a prosthetic heart valve 200 comprising a radially expandable and compressible annular frame 202 and a plurality of leaflets 204 secured to the frame. Each leaflet 204 can comprise opposing commissure tabs 250 disposed on opposite sides of the leaflet 204 and a cusp edge portion 252 extending between the opposing commissure tabs. As described further below, the cusp edge portion 252 of each leaflet 204 can have an inflow end 226 (or edge, disposed at a central portion of the cusp edge portion 252) that is arranged at an inflow end of the prosthetic heart valve (for example, inflow end 212 of a frame 202 of the prosthetic heart valve 200).

The frame 202 can be made of any of various suitable plastically-expandable materials (for example, stainless steel, etc.) or self-expanding materials (for example, Nitinol). In some examples, the frame 202 comprises a plastically-expandable material, such as those described above with reference to the prosthetic heart valve 10 of FIG. 1.

The frame 202 can comprise a plurality of interconnected struts 206 which form multiple rows of open cells 208 between an outflow end 210 and an inflow end 212 of the frame 202. In some examples, as shown in FIG. 3, the frame 202 can comprise three rows of cells 208 with a first (for example, upper in FIG. 3) row of cells 214, disposed at the outflow end 210, having cells 208 that are elongated in an axial direction (relative to a central longitudinal axis 216 of the frame 202), as compared to cells 208 in the remaining rows of cells. For example, the cells 208 of the first row of cells 214 can have a longer axial length, defined in a direction of the central longitudinal axis 216 of the frame 202, than cells 208 in the remaining rows of cells (for example, cells in the row of cells at the inflow end 212).

In some examples, as shown in FIG. 3, each row of cells 208 comprises nine cells. Thus, in such examples, the frame 202 can be referred to as a nine-cell frame.

In other examples, the frame 202 can comprise more than three rows of cells (for example, four or five) and/or more or less than nine cells per row. In some examples, the cells 208 in the first row of cells 214 may not be elongated compared to cells 208 in remaining rows of cells of the frame 202.

The interconnected struts 206 can include a plurality of angled struts 218, 234, 236, and 238 arranged in a plurality of rows of circumferentially extending rows of angled struts, with the rows being arrayed along the length of the frame between the outflow end 210 and the inflow end 212 of the frame 202. For example, the frame 202 can comprise a first row of angled struts 238 arranged end-to-end and extending circumferentially at the inflow end 212 of the frame; a second row of circumferentially extending, angled struts 236; a third row of circumferentially extending, angled struts 234; and a fourth row of circumferentially extending, angled struts 218 at the outflow end 210 of the frame 12. The fourth row of angled struts 218 can be connected to the third row of angled struts 234 by a plurality of axially extending window struts or struts 240 and a plurality of axial (for example, axially extending) struts 232. The axially extending window struts 240 (which can also be referred to as “axially extending posts” and “axially extending struts”) define commissure windows (for example, open windows) 242 that are spaced apart from one another around the frame 202, in a circumferential direction, and which are adapted to receive a pair of commissure tabs of a pair of adjacent leaflets 204 arranged into a commissure 230. The window struts 240 forming the commissure windows 242 can also be referred to herein as commissure supports of the frame 202.

One or more (for example, two, as shown in FIG. 3) axial struts 232 can be positioned between, in the circumferential direction, two commissure windows 242 formed by the window struts. Since the frame 202 can include fewer cells per row (for example, nine) and fewer axial struts 232 between each commissure window 242, as compared to other prosthetic heart valves, such as the prosthetic heart valve 10 of FIG. 1, each cell 208 can have an increased width (in the circumferential direction), thereby providing a larger opening for blood flow and/or coronary access, as described herein.

Each axial strut 232 and each window strut 240 extends from a location defined by the convergence of the lower ends (for example, ends arranged inward of and farthest away from the outflow end 210) of two angled struts 218 (which can also be referred to as an upper strut junction or upper elongated strut junction) to another location defined by the convergence of the upper ends (for example, ends arranged closer to the outflow end 210) of two angled struts 234 (which can also be referred to as a lower strut junction or lower elongate strut junction). Each axial strut 232 and each window strut 240 forms an axial side of two adjacent cells of the first row of cells 214.

In some examples, as shown in FIG. 3, each axial strut 232 can have a width 244 that is larger than a width of the angled struts 218, 234, 236, and/or 238. As used herein, a “width” of a strut is measured between opposing locations on opposing surfaces of a strut that extend between the radially facing inner and outer surfaces of the strut (relative to the central longitudinal axis 216 of the frame 202). A “thickness” of a strut is measured between opposing locations on the radially facing inner and outer surfaces of a strut and is perpendicular to the width of the strut. In some examples, the width 244 of the axial struts 232 is 50-200%, 75-150%, or at least 100% larger than (for example, double) the width of the angled struts of the frame 202. For example, if the angled struts 218, 234, 236, and 238 are approximately 0.3 mm wide, then the width 244 of the axial struts 232 can be in a range of 0.45 mm-0.9 mm, 0.5 mm-0.75 mm, or at least 0.6 mm.

For example, in known prosthetic heart valves (such as the valve 10 shown in FIG. 1) the axial struts have a same width as the other struts (for example, angled struts) of the frame. As such, when the leaflets of the known prosthetic heart valve are pressed against the frame during the systolic phase, the leaflets may bend around the struts and/or extend radially outwardly through the cell openings due to the relatively narrow width of the axial struts. This phenomenon can reduce long-term durability of the leaflets, especially when the upper (for example, outflow/free) edges of the leaflets are pushing against the axial struts and are bent over, as described above.

Thus, by providing the axial struts 232 with the width 244 that is greater than the width of other struts (for example, angled struts of the frame 202), a larger contact area is provided for when the leaflets 204 contact the wider axial struts 232 during systole. This can, for example, distribute the stress and reduce the extent to which the leaflets 204 fold over the axial struts 232 and/or extend radially outward through the cells 208. As a result, one advantage of the disclosed technology is that the long-term durability of the leaflets 204 is improved.

In some cases, the free edges at the outflow end 228 of the leaflets 204 may press against the axial struts 232 at their outflow (for example, upper) end portions 246. Accordingly, in some examples, the outflow end portions 246 of the axial struts 232 can be even wider than the width 244, which is depicted at an intermediate location of the axial strut 232 in FIG. 3, thereby providing an even larger area of contact and support for the leaflets 204.

As introduced above, since the frame 202 can have fewer cells in the circumferential direction (for example, nine in the example depicted in FIG. 3) compared to the prosthetic valve of FIG. 1, each cell 208 can have an increased width (measured in the circumferential direction). This increased width of the cells 208 of the first row of cells 214 can enable the wider axial struts 232 to be incorporated into the frame 202, without sacrificing open space for blood flow and/or coronary access.

Commissure tabs 250 of adjacent leaflets 204 can be secured together to form commissures 230. Each commissure 230 of the prosthetic heart valve 200 comprises two commissure tabs 250 paired together, one from each of two adjacent leaflets 204, and extending through a commissure window 242 of the frame 202. Each commissure 230 can be secured to the window struts forming the commissure window 242.

The cusp edge portion (for example, scallop edge) 252 of each leaflet 204 can be secured to the frame via one or more fasteners (for example, sutures). In some examples, as shown in FIG. 3, the cusp edge portion of each leaflet 204 can be secured directly to the struts of the frame 202 (for example, angled struts 234, 236, and 238). For example, the cusp edge portions 252 of the leaflets 204 can be sutured to the angled struts 234, 236, 238 that generally follow the contour of the cusp edge portions of the leaflets.

In some examples, the cusp edge portion 252 of the leaflets 204 can be secured to an inner skirt and the inner skirt can then be secured directly to the frame 202.

Further, in some examples, an outer skirt can be connected to an outer surface of the frame 202 (for example, similar to the outer skirt 18 of the valve 10 of FIG. 1).

As shown in FIG. 3, in some examples, one or more of or each of the axial struts 232 can comprise an inflow end portion (for example, inflow end portion that is closer to the inflow end than the outflow end portion 246) 248 that is widened relative to a middle portion 247 of the axial strut 232 (which can be defined by the width 244), similar to the outflow end portion 246 (as described above). In some examples, the inflow end portion 248 of the axial strut 232 can comprise an aperture 249. The apertures 249 can be configured to receive fasteners (for example, sutures) for attaching soft components of the prosthetic heart valve 200 to the frame 202. For example, in some examples, an outer skirt can be positioned around an outer surface of the frame 202 and secured to the apertures 249 (for example, as shown in FIGS. 31 and 32 and described further below).

The frame 202 can further comprise a plurality of apices 220 formed at the inflow end 212 and the outflow end 210, each apex 220 forming a junction between two angled struts 218 at the inflow end 212 or outflow end 210. As such, the apices 220 are spaced apart from one another, in a circumferential direction at the inflow end 212 and the outflow end 210. Each apex 220 can have a height 221.

In some examples, as shown in FIG. 3, each apex 220 can have side portions 222 that curve or bend axially outward from the angled strut 218 to which it is connected and an end portion 224 that extends between the two side portions 222 of the apex 220. The side portions 222 can extend in a direction that is parallel to the central longitudinal axis 216. The end portion 224 can be relatively flat and include a surface that is disposed normal to the central longitudinal axis 216. Each apex 220 can have two bends at its end portion 224 and two bends at the side portions 222 (for example, one at the junction between each side portion 222 and angled strut 218). In this way, the apices 220 can be U-shaped, similar to the apices 26 of the valve of FIG. 1. However, in other examples, the apices 220 can have another shape such as a more curved and longer apex region with a reduced height in the axial direction (for example, as shown in FIGS. 20-32), such as disclosed in U.S. Provisional Application Nos. 63/178,416 and 63/194,830, both of which are incorporated herein by reference.

As introduced herein, a leaflet assembly of a prosthetic heart valve comprising a plurality of leaflets (for example, leaflets 204 shown in FIG. 3) can either be secured to an inner skirt which is then secured to the struts of the frame of the prosthetic heart valve or directly to the struts of the frame (without the inner skirt). An example of directly securing the leaflets 204 to the struts 206 of the frame 202 is shown in FIG. 3. Reducing the number of soft components of the prosthetic heart valve (for example, by eliminating the inner skirt and suturing the leaflets directly to the frame) can simplify a process for assembling the prosthetic heart valve. For example, assembling prosthetic heart valves including an inner skirt can result in a prolonged assembly time which involves suturing each of the leaflets to the inner skirt and then suturing the inner skirt to the frame of the prosthetic heart valve.

Leaflet commissures, such as those described above, may be subject to various stresses both during the delivery of a prosthetic heart valve and after the prosthetic heart valve is deployed in a patient's vasculature. For example, the frame may apply stresses to the leaflet during radial expansion of the frame, and the opening and closing of the valvular structure under the flow of blood may further stress the leaflets. Particularly, when load-bearing sutures are passed through the leaflets, they provide stress concentrators in the leaflet material. To mitigate stresses on the leaflets at the commissures, known prosthetic valves can include multiple layers of leaflet material and reinforcing fabric forming each commissure. While durable, the various layers of material forming the commissures can limit the crimp profile of the prosthetic valve. Disclosed herein are examples of commissures that include reinforcing members stitched to respective commissure tabs along two or more suture lines, wherein each commissure tab can be secured to a frame portion with the main load bearing sutures in the form of stitches that extend through respective stitches of two suture lines, rather than through the leaflet itself. Advantageously, this removes stress concentrators from the leaflet material, thereby permitting the use of fewer layers of material to form the commissures. This, in turn, decreases the crimp profile (that is, the diameter of the prosthetic heart valve in the radially compressed configuration) of the prosthetic heart valve. In this way, a commissure assembly with a narrow, yet sturdy configuration is provided.

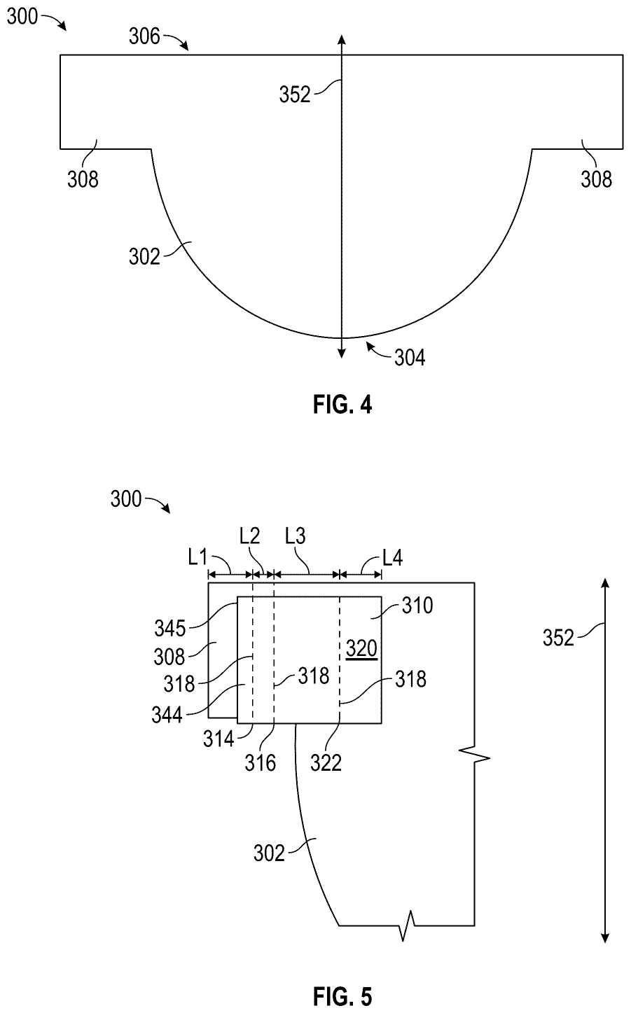

FIG. 4 is a front view in elevation of an example leaflet 300 that shares some similar characteristics with and may be used in the same applications as the leaflet 204 described above. The leaflet 300 has a leaf-shaped leaflet body 302 with an inflow end portion (or edge) 304 and an outflow end portion (or edge) 306, with an axis 352 extending between the inflow end portion 304 and the outflow end portion 306. The leaflet 300 has at least one commissure tab, and typically two opposing commissure tabs 308 as shown. The commissure tabs 308 extend from the opposing lateral sides of the outflow end portion 306. When attached to a radially expandable frame, such as the frame 12 described above, at least one (and typically two) of the commissure tabs 308 can extend through a commissure window (for example, the commissure window 242 defined by window struts or struts 240 as shown in FIG. 3). Each commissure window can accommodate a pair of commissure tabs 308 of two adjacent leaflets 300 (sometimes referred to as two tabs or a pair of tabs), such that the adjacent leaflets 300 form a commissure between them, as described above in relation to the prosthetic heart valve 200.

The valvular structure of a prosthetic heart valve can, in this way, be formed from a plurality of leaflets 300 joined at the commissure tabs 308 and secured in a plurality of commissure windows, in the same or substantially the same way as the valvular structure 14, except for the differences described herein.

FIG. 5 is a front view of a portion of the leaflet 300 of FIG. 4 with a reinforcing member 310 attached to one of the commissure tabs 308 of the leaflet 300. The reinforcing member 310 can cover all or part of the commissure tab 308. A commissure formed from a pair of adjacent leaflet 300s can comprise two reinforcing members 310, each covering a commissure tab 308 of a respective leaflet (only one reinforcing member is in shown in FIG. 5, which depicts only a portion of one leaflet 300).

In some examples, the reinforcing member 310 can be made of any of various biocompatible fabrics, such as polyethylene terephthalate (PET) fabric, or non-fabric materials, such as polyurethane, silicone, or other polymers. In some examples, the reinforcing member 310 can be made of natural tissue, such as pericardium. In some examples, the reinforcing member 310 can comprises a combination of natural tissue and a synthetic material.

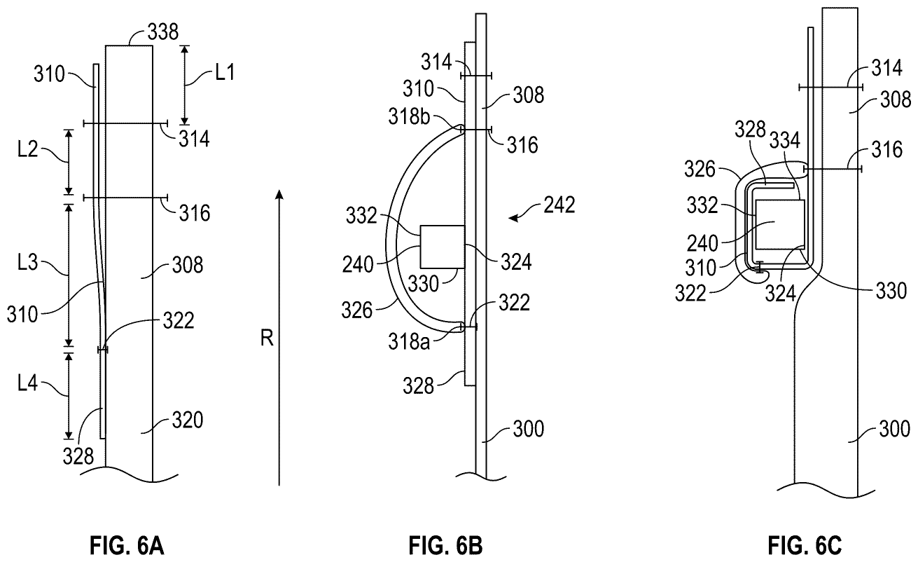

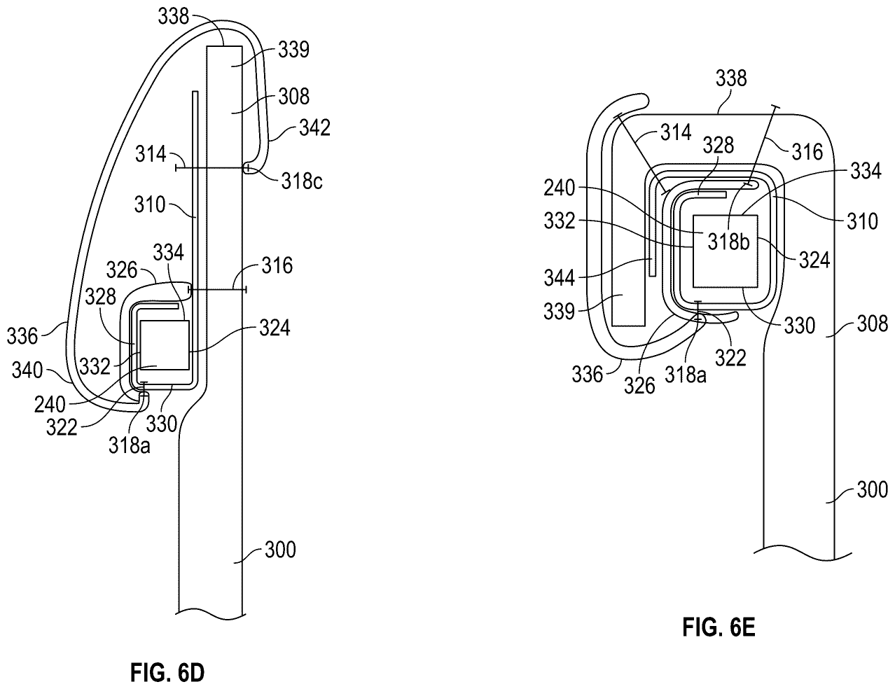

The reinforcing member 310 can be attached to the leaflet 300 by one or more suture lines (also referred to as stitch lines). For example, as shown in FIG. 5, a first suture line 314 and a second suture line 316 can extend through the reinforcing member 310 and the commissure tab 308, thereby securing the reinforcing member 310 to the commissure tab 308. The first suture line 314 and the second suture line 316 can each form a plurality of in-and-out stitches 318 that extend out of a face 320 of the reinforcing member 310, as well as out of an opposite face of the commissure tab 308 (FIG. 6A). Thus, the first suture line 314 and the second suture line 316 can provide one or more attachment points for various connecting sutures, as further described herein. In some examples, as shown in FIG. 5, the stitches 318 of the first suture line 314 are staggered or offset from the stitches 318 of the second suture line 316 in the direction of axis 352.

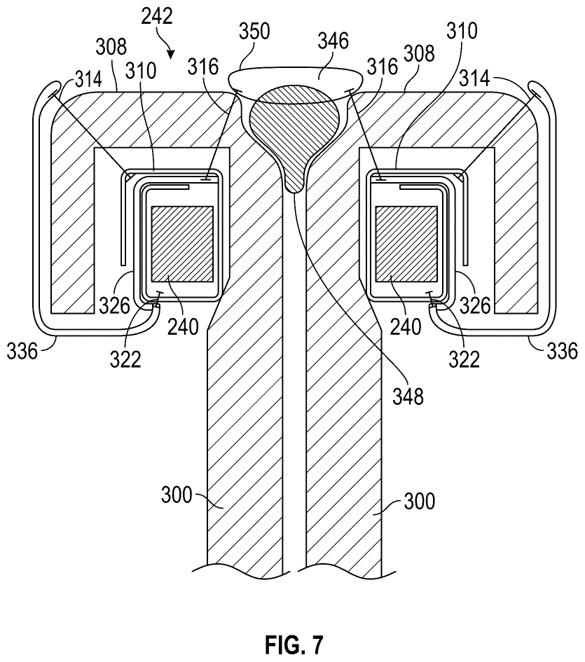

Turning now to FIG. 6A, which shows a top plan view of the portion of the leaflet 300 and the attached reinforcing member 310 of FIG. 5, the first suture line 314 can be offset from a side edge 338 of the commissure tab 308 by a distance L1. The distance L1 can be selected such that the side edge 338 of the commissure tab 308 can extend fully along one face of a window strut 240 while positioning the first suture line 314 along or around a corner between one face of the window strut 240 and an adjacent face of the window strut 240 (for example, as shown in FIGS. 6E and 7). Accordingly, the distance L1 can vary according to the geometry of the window strut 240. In some examples, the distance L1 can be between 0.6 mm and 1.0 mm, or in particular examples, about 0.8 mm. As also shown in FIG. 6A, the edge of the reinforcing member 310 can be offset from the side edge 338 of the commissure tab.

As also shown in FIG. 6A, the first suture line 314 and the second suture line 316 can be spaced apart from one another along the length of the reinforcing member 310 by a distance L2. The distance L2 between the first suture line 314 and the second suture line 316 can be selected such that, when the commissure tab and reinforcing member 310 are folded around the window strut 240, the first suture line 314 and the second suture line 316 are positioned proximate to opposite ends of a single face of the window strut 240 (for example, as shown in FIGS. 6E and 7). Advantageously, this configuration can limit or prevent the rotation of the reinforcing member 310 and the commissure tab 308 around the window strut 240. Accordingly, the distance L2 can vary according to the geometry of the window strut 240. In some examples, the distance L2 can be between 0.4 mm and 0.8 mm. In some specific examples, the distance L2 is about 0.6 mm.

Returning to FIG. 5, the leaflet 300 can also include a third suture line 322. The third suture line 322 can extend solely through the reinforcing member 310 (that is, it can pass through the reinforcing member 310 and not through the outflow end portion of the leaflet body 302 or through the commissure tab 308), as shown in FIG. 6A. The third suture line 322 can form a plurality of in-and-out stitches 318 that extend out of the face 320 of the reinforcing member 310, and a corresponding plurality of stitches 318 that extend out of the opposite face of the reinforcing member 310 (that is, between the reinforcing member 310 and the commissure tab 308) as shown in FIG. 6A. Thus, the third suture line 322 can provide one or more attachment points for various connecting sutures, as further described herein.

As shown in FIG. 6A, the third suture line 322 can be spaced apart from the second suture line 316 by a distance L3. The distance L3, in some examples, can be selected to position the second suture line 316 and the third suture line 322 on opposing faces of the window strut 240 (as shown in FIG. 6C). Accordingly, the distance L3 may vary depending on the geometry of the window strut 240. In some examples, the distance L3 can be 0.5 mm to 1.5 mm. In some specific examples, the distance L3 is between 0.8 mm and 1.2 mm, or more particularly about 1.0 mm.

The third suture line 322 may also be spaced apart from an inner side edge of the reinforcing member 310 by a distance LA. The distance LA can be selected such that a sufficient length of the reinforcing member 310 extends past the third suture line 322 to form an end portion 328 that, when wrapped around the window strut 240, extends fully across at least one face of the window strut 240 (for example, as shown in FIG. 6C). Accordingly, the distance L4 may vary depending on the geometry of the window strut 240. In some examples, the distance L4 can be between 0.4 mm and 0.8 mm. In some specific examples, the distance L4 is about 0.6 mm.

The arrangement of the commissure tab 308, the reinforcing member 310, and the suture lines 314, 316, 322 allows the leaflet 300 to be attached to a commissure window (for example, commissure window 20 of frame 12 or commissure window 242 of frame 202) with minimal need for additional leaflet material to reinforce attachment points, while avoiding load-bearing stitches that extend through the leaflet or at least minimizing the number of load-bearing stitches that extend through the leaflet, as described in greater detail herein. An example attachment of a commissure tab 308 of the leaflet 300 to a window strut 240 of a commissure window 242 is illustrated in FIGS. 6B-6E. It will be appreciated that FIGS. 6B-6E are not drawn to scale to improve clarity in the depiction of the interaction between the components and/or the arrangement of the components, such as the leaflet 300, the reinforcing member 310, the suture lines 314, 316, and 322, and the window strut 240 of the commissure window 242.

While the examples described herein pertain to a commissure window formed by two rectangular window struts, such as window struts, it is understood that the geometry of the window struts can be varied according to the needs of the frame. For example, the window struts can have a circular, ovular, or polygonal cross section. In such examples, the reinforcing member 310 and the commissure tab 308 can be wrapped or folded around the circumference, perimeter, or any number of sides of the window struts.

As also described above, FIG. 6A shows a top plan view of a leaflet assembly comprising the leaflet 300 and the reinforcing member 310, positioned adjacent to one another, with the reinforcing member 310 located along the outflow end portion, and the first and second suture lines 314, 316 extending through both the reinforcing member 310 and the leaflet 300. The third suture line 322 extends through the reinforcing member 310, but in some examples such as the one shown, does not extend through the leaflet 300.

To attach the commissure tab 308 of the leaflet 300 and the reinforcing member 310 to a window strut 240 of a commissure window 242, the leaflet 300 and the reinforcing member 310 can be inserted through the commissure window 242 from inside the frame 202 and can extend radially outward (that is, further in the direction indicated by the arrow R of FIG. 6B) past the window strut 240, as shown in FIG. 6B, such that a portion of the commissure tab 308 and the reinforcing member are outside the frame 202. In the example shown, the first suture line 314 and the second suture line 316 are positioned radially outwards of the window strut 240 or the commissure window 242 (outside of the frame 202), and the third suture line is positioned radially inwards of the window strut 240 (inside the frame 202). The reinforcing member 310 can be positioned against a first face 324 of the window strut 240. For purposes of illustration, only one window strut 240 of the window 242 is shown in FIGS. 6B-6E.

With continued reference to FIG. 6B, a first securing loop 326 (also referred to herein as a first suture loop) can be formed to extend between the third suture line 322 and the second suture line 316. The first securing loop 326 can pass through a first stitch 318a in the third suture line 322 and through a second stitch 318b in the second suture line 316. As shown in FIG. 6B, the first securing loop 326 can extend around the window strut 240, such that the window strut 240 is located between the first securing loop 326 and the reinforcing member 310. In some examples, the first securing loop 326 can extend only through a single stitch 318a of the third suture line 322 and a single stitch 318b of the second suture line 316. In some examples, the first securing loop 326 can extend through multiple stitches 318a and/or multiple stitches 318b, such that the first securing loop 326 forms multiple loops extending continuously back and forth between the second suture line 316 and the third suture line 322 along the length of the suture lines 316, 322 in a helical or zig-zag pattern. In some examples, a plurality of discrete securing loops 326 can be formed, each of which extends through a respective pair of stitches 318a, 318b along the length of the suture lines 316, 322.

When the first securing loop 326 is tightened, as illustrated in FIG. 6C, the first end portion 328 of the reinforcing member 310 is pulled away from the leaflet 300 as the first securing loop pulls on the third suture line 322. As the first securing loop 326 is tightened further, the first end portion 328 of the reinforcing member 310 is then, in the case of a window strut 240 with a rectangular cross section as shown in FIGS. 6A-6E, pulled around a second face 330, a third face 332, and at least partially around a fourth face 334 of the window strut 240. As the securing loop is tightened further, the reinforcing member 310 is pulled more tightly against the faces 324, 330, 332, and 334 of the window strut 240, with the third suture line 322 adjacent the second face 330 of the window strut 240. In this way, when the first securing loop 326 is tightened, the reinforcing member 310 is folded to at least partially enclose the window strut 240 and the reinforcing member 310.

Turning now to FIG. 6D, a second securing loop 336 (also referred to herein as a second suture loop) can be added, which is formed to extend between the third suture line 322 and the first suture line 314. The second securing loop 336 can pass through a first stitch 318a in the third suture line 322 and through a third stitch 318c in the first suture line 316. As shown in FIG. 6D, the second securing loop 336 can extend around the window strut 240, such that the window strut 240 is located between the second securing loop 336 and the commissure tab 308. The second securing loop 336 also extends around an end portion 339 of the commissure tab 308, such that a first end portion 340 and a second end portion 342 of the second securing loop 336 are positioned on opposite sides of the leaflet 300.

In some examples, the second securing loop 336 can extend only through a single stitch 318a of the third suture line 322 and a single stitch 318c of the first suture line 314. In some examples, the second securing loop 336 can extend through multiple stitches 318a and/or multiple stitches 318c, such that the second securing loop 336 forms multiple loops extending continuously back and forth between the first suture line 314 and the third suture line 322 along the length of the suture lines 314, 322 in a helical or zig-zag pattern. In some examples, a plurality of discrete securing loops 336 can be formed, each of which extends through a respective pair of stitches 318a, 318c along the length of the suture lines 314, 322.

When the second securing loop 336 is tightened, as illustrated in FIG. 6E, a second end portion 344 of the reinforcing member 310 is folded inwards around the window strut 240. The second end portion 344 can extend along the length L1 (shown in FIG. 5), between the first suture line 314 and an end 345 of the reinforcing member 310. In some examples, the second end portion 344 of the reinforcing member 310 may include a portion of the reinforcing member 310 positioned about the end portion 339 of the commissure tab 308 and extending distally from the first suture line 314.

As shown in FIG. 6E, the second end portion 344 is folded around the fourth face 334 and the third face 332 of the window strut 240, such that the second end portion 344 folds around the first end portion 328 of the reinforcing member 310 and the first securing loop 326. In an example shown in FIG. 6E, the reinforcing member 310 can fold or wrap entirely around the window strut 240. Also, the reinforcing member 310 can, when the second securing loop 336 is fully tightened, compress and secure the first securing loop 326 between the first end portion 328 and the second end portion 344 of the reinforcing member 310, preventing slippage and/or loosening of the first securing loop 326 and fastening the reinforcing member 310 to the window strut 240. In some examples, the reinforcing member 310 may not fold or wrap entirely around the window strut 240.

With continued reference to FIG. 6E, tightening the second securing loop 336 also folds and secures the end portion 339 of the commissure tab 308 inwards around the window strut 240 and around the reinforcing member 310. For instance, in the illustrated example having a window strut 240 with a rectangular cross section, when the second securing loop 336 is tightened, the commissure tab 308 is pulled securely around the first face 324, the fourth face 334, and the third face 332 of the window strut 240. In some examples, such as the one illustrated in FIG. 6E, the commissure tab 308 can thus extend parallel to or substantially parallel to at least the first face 324, the fourth face 334, and the third face 332 of the window strut 240. In this way, when the second securing loop 336 is tightened, a portion of the first leaflet (for example, the commissure tab 308) is folded to at least partially enclose the window strut 240 and the reinforcing member 310.

Thus, the reinforcing member 310 is secured between the commissure tab 308 and the window strut 240, and the commissure tab 308 extends along a portion or all of at least three faces (324, 332, 334) of the window strut 240. In some examples, the bends in the commissure tab 308 can be right angle bends or approximately right-angle bends (that is, the bends can each be 90-degree bends or approximately 90-degree bends).

A detailed view of a commissure formed from two adjacent leaflets with their commissure tabs extending through a commissure window is shown in FIG. 7. Specifically, FIG. 7 shows that the commissure window 242 defined by the two axially extending window struts 240 can receive the two commissure tabs 308 of the two adjacent leaflets 300. As previously described, each leaflet 300 can have the reinforcing member 310 attached by the first suture line 314 and the second suture line 316, as well as the third suture line 322. Each commissure tab 308 can be secured to a respective window strut 240, as previously described in connection with FIGS. 6A-6E. Thus, to attach a pair of commissure tabs to a frame of a prosthetic valve, a first commissure tab 308 and corresponding reinforcing member 310 can be secured to a window strut 240 as shown in FIGS. 6A-6E and described above, and then a second commissure tab 308 and corresponding reinforcing member 310 can be inserted through the window 242 and secured to the other window strut 240 of the window 242.

To further secure the commissure tabs 308 to the window struts 240, a commissure wedge member 346 can optionally be positioned between the adjacent commissure tabs 308. The commissure wedge member 346 can comprise a wedge head 348 that extends (for example, extending at least along a portion of the height of the commissure tabs 308) between the adjacent commissure tabs 308. When the commissure wedge member 346 is positioned between the adjacent commissure tabs 308, the commissure tabs 308 are compressed between the window struts and the commissure wedge member 346, preventing slippage of the commissure tabs 308 relative to the window struts. The wedge member 346 can comprise, for example, a piece of fabric or a relatively thick suture (for example, a braided suture, such as an Ethibond suture).

A third securing loop 350 (that is, a third suture loop or wedge securing loop 350) can extend from the second suture line 316 of one commissure tab 308 to the second suture line 316 of the other commissure tab 308. The third securing loop 350 can extend radially outwards around the commissure wedge member 346, and optionally through the wedge member 346. When the third securing loop 350 is tightened, the commissure wedge member 346 can be pressed radially inwards against the commissure tabs 308, further compressively securing the commissure tabs 308.

In some examples, a prosthetic heart valve, such as the prosthetic heart valve 10 and the prosthetic heart valve 200 comprises multiple commissure windows 242 for multiple leaflets 300, forming a corresponding number of commissures assembled in the fashion described above in relation to FIGS. 6A through 7. For example, a prosthetic heart valve can have two or more leaflets 300, such as three leaflets 300, or more than three leaflets 300, wherein each leaflet 300 comprises two commissure tabs 308, and a matching number of commissure windows 242 receive the commissure tabs 308 to form a number of commissures equal to the number of leaflets, such as two commissures 230, three commissures 230, or more than three commissures 230.

In this way, one or more relatively narrow commissure assemblies that are nonetheless durable can be assembled. Because the tensioning forces securing the commissure tabs 308 to the window strut are borne at least by the first securing loop 326 and/or the second securing loop 336, and because the first securing loop 326 and the second securing loop 336 do not pass through the leaflet 300, the securing loops 326, 336 do not introduce any holes or apertures in the leaflet 300. Accordingly, no stress concentrators are added to the leaflet, thereby improving leaflet durability.

Also, because no suture line attaches any leaflet to itself or to another leaflet, each of the window struts 240 needs only accommodate a single layer of leaflet material (that is, a single commissure tab 308 folded around the window strut 240), the assembly comprising the commissure window 242, the commissure tabs 308, and the reinforcing members 310 as described herein, for example, may have a reduced profile compared to known prosthetic valves. This, in turn, reduces the overall crimp profile of the prosthetic heart valve.

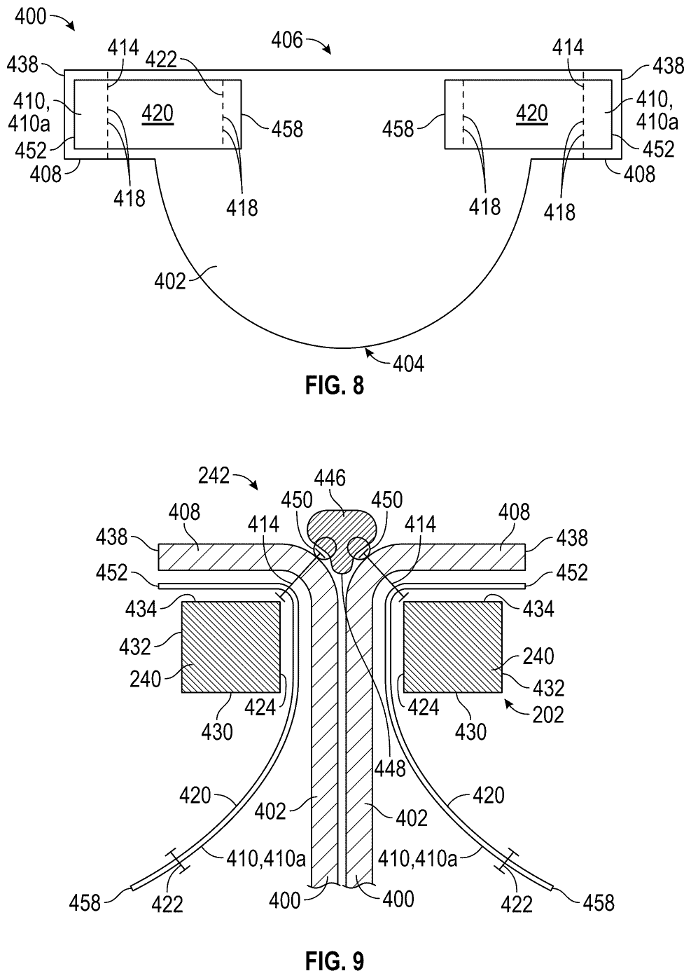

Additional exemplary leaflets including attached reinforcement members, exemplary stages of commissure attachment, and exemplary commissure configurations are shown in FIGS. 8-15. It will be appreciated that, in some examples, the exemplary leaflets, reinforcing members, and commissure attachment configurations of FIGS. 8-15 can have one or more features of the examples described above with respect to FIGS. 1-7. In some examples, the exemplary leaflets, reinforcing members, and commissure attachment configurations of FIGS. 8-15 can have one or more different features relative to the examples of FIGS. 1-7, as described below.

Turning to FIG. 8, a front view in elevation of an example leaflet 400 including attached reinforcing members 410 is shown and described. In some examples, the leaflet 400 can have similar characteristics to and may be used in the same or similar applications as the leaflets 204, 300, described above. For example, the leaflet 400 can include a leaf-shaped leaflet body 402 with an inflow end portion (or edge) 404 and an outflow end portion (or edge) 406 and opposing commissure tabs 408 for attachment to a commissure window of a frame (such as, for example, the commissure window 242 of the frame 202 shown in FIG. 3 or other commissure windows). The reinforcing members 410 can each be attached to one of the commissure tabs 408. The commissure tabs 408 can be paired with commissure tabs of adjacent leaflets, thereby forming a valvular structure (which can be similar to, for example, the valvular structure 14 of FIG. 1 and/or the valvular structure formed from the leaflets 204 shown in FIG. 3).

As shown in FIG. 8, in some examples, the reinforcing members 410 can each cover a portion of the respective commissure tab 408. In some examples, the reinforcing members 410 can each cover an entirety of the respective commissure tab 408. In some examples, the reinforcing members 410 can each be centered, in an axial direction, relative to the respective commissure tab 408. In some examples, the reinforcing members 410 can each be offset in an axial direction relative to the respective commissure tab 408 (similar to the offset configuration of the reinforcing member 310 and the commissure 308 shown in FIG. 5).

In some examples, the reinforcing members 410 can be made of any of various biocompatible fabrics, such as polyethylene terephthalate (PET) fabric, or non-fabric materials, such as polyurethane, silicone, or other polymers. In some examples, the reinforcing members 410 can be made of natural tissue, such as pericardium. In some examples, the reinforcing members 410 can comprises a combination of natural tissue and a synthetic material.

The reinforcing members 410 can be attached to the leaflet 400 by a single suture line (also referred to as stitch line). As shown in FIG. 8, a first suture line 414 extends through each of the reinforcing members 410 and the commissure tabs 408, thereby securing the reinforcing members 410 to the respective commissure tab 408. In some examples, the first suture lines 414 can be similar to the first suture line 314. For example, each of the first suture lines 414 can form a plurality of in-and-out stitches 418 that extend out of a face 420 of the reinforcing member 410, as well as out of an opposite face of the commissure tab 408. In the illustrated example, different from the leaflet 300 and the reinforcing member 310, only one suture line (the suture line 414) extends through the reinforcing member 410 and the commissure tab 408. In other words, a suture line similar to the suture line 316 is excluded from the leaflet 400.

In some examples, the leaflet 400 can include second suture lines 422. Each of the second suture lines 422 can extend solely through the respective reinforcing member 410 (that is, similar to the third suture line 322, it can pass through the reinforcing member 410 and not through the outflow end portion of the leaflet body 402 or the commissure tab 408). The second suture lines 422 can form a plurality of in-and-out stitches 418 that extend out of the face 420 of the respective reinforcing member 410, and a corresponding plurality of stitches 418 that extend out of the opposite face of the reinforcing member 410 (that is, between the reinforcing member 410 and the commissure tab 408). Each of the first suture lines 414 and the second suture lines 422 can provide one or more attachment points for various connecting sutures (also referred to herein as “securing loops”).

In some examples, as described above for the suture lines 314, 322, the locations of the suture lines 414, 422 on the reinforcing members 410 can be selected based geometries and/or sizes of the struts 240, the commissure window 242, the commissure tabs 408, and/or a desired commissure configuration. In some examples, a length of the reinforcing members 410 and/or a position of the reinforcing members 410 relative to the commissure tabs 408 can be selected based geometries and/or sizes of the struts 240, the commissure window 242, the commissure tabs 408, and/or a desired commissure configuration. In some examples, a side edge 438 of the commissure tab 408 can extend past a side edge 452 of the reinforcing member 410 (for example, as in the reinforcing member 410a shown in FIGS. 8-10). In some examples, the side edge 452 of the reinforcing member 410 can extend past the side edge 438 of the commissure tab 408 (for example, as in the reinforcing member 410b shown in FIG. 11).

The arrangement of the commissure tabs 408, the reinforcing members 410, and the suture lines 414, 422 can allow the leaflets 400 to be attached to a commissure window (for example, commissure window 20 of frame 12 or commissure 242 of frame 202) with minimal need for additional leaflet material to reinforce attachment points, while avoiding load-bearing stitches that extend through the leaflet or at least minimizing load-bearing on stitches that extend through the leaflet, as described in greater detail herein. Further, the reduced number of suture lines relative to the leaflet 300 (including the reinforcing members 310) simplifies assembly of the leaflet 400, while still preventing and/or minimizing load-bearing on stitches that extend through the leaflet.

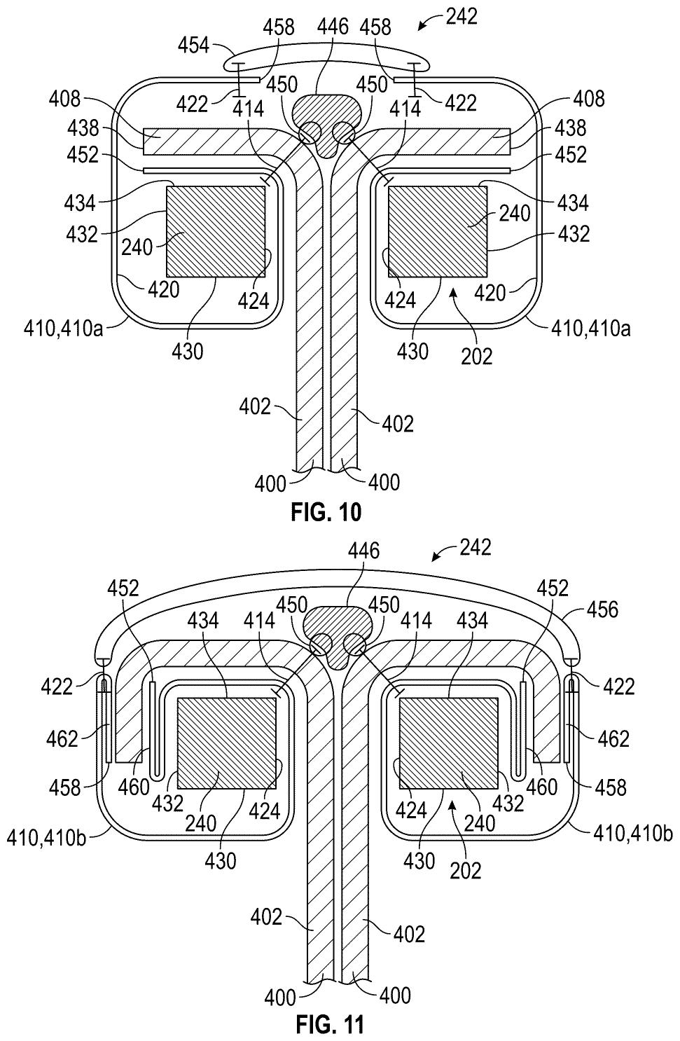

An exemplary first stage of attachment of paired commissure tabs 408 of adjacent leaflets 400 to window struts 240 of a commissure window 242 is illustrated in FIG. 9. An example second stage of attachment of the paired commissure tabs 408 of adjacent leaflets 400 to the strut 240 of the commissure window 242 is illustrated in FIG. 10, and shows a first exemplary configuration for a commissure that can be formed with the leaflets 400 including reinforcing members 410a. A second exemplary configuration for a commissure that can be formed with the leaflets 400 and including reinforcing members 410b is shown in FIG. 11. It will be appreciated that FIGS. 9-11 are not drawn to scale to improve clarity in the depiction of the interaction between the components and/or the arrangement of the components, such as the leaflet 400, the reinforcing members 410, the suture lines 414, 422, the window struts 240 of the commissure window 242, and the connecting sutures (discussed below).

To attach the commissure tabs 408 of the leaflets 400 and the reinforcing members 410 to a window strut 240 of a commissure window 242, the commissure tabs 408 and the reinforcing members 410 can be inserted through the commissure window 242 from inside the frame 202 and can extend radially outward past the window strut 240 such that a portion of each commissure tab 408 and reinforcing member 410 is outside the frame 202. As shown in FIG. 9, at the first stage of formation, the first suture lines 414 can be positioned radially outwards of the window struts 240 and the commissure window 242 (outside of the frame 202), and the second suture lines 422 can be positioned radially inwards of the window struts 240 and the commissure window 242 (inside the frame 202). The commissure tabs 408 and reinforcing members 410 can extend over first faces 424 of the window struts 240. The reinforcing members 410 can be positioned against the first face 424 of each of the window struts 240 and can be disposed between the respective commissure tab 408 and the fourth face 434. In some examples, the bends in the commissure tabs 408 can be right angle bends or approximately right-angle bends (that is, the bends can each be 90-degree bends or approximately 90-degree bends).

In the examples shown in FIGS. 9 and 10, portions of the reinforcing members 410a and the commissure tabs 408 exterior of the frame 202 can be wrapped over the struts 240 to extend over fourth (exterior) faces 434 thereof. The reinforcing members 410a can be positioned against the fourth face 434 and can be disposed between the respective commissure tab 408 and the fourth face 434 of the strut 240. When extending over the fourth faces 434 of the struts 240, a side edge 438 of each commissure tab 408 can be generally aligned with the side edge 452 of the reinforcing member 410a attached thereto.

To secure the commissure tabs 408 to the window struts 240, a commissure wedge member 446 can be positioned between the adjacent commissure tabs 408 at a location where they exit the commissure window 242. The commissure wedge member 446 can comprise a wedge head 448 that extends (for example, extending at least along a portion of the height of the commissure tabs 408) between the adjacent commissure tabs 408. When the commissure wedge member 446 is positioned between the adjacent commissure tabs 408, the commissure tabs 408 are compressed between the window struts 240 and the commissure wedge member 446, preventing slippage of the commissure tabs 408 relative to the window struts 240. The wedge member 446 can comprise, for example, a piece of fabric or a relatively thick suture (for example, a braided suture, such as an Ethibond suture).

In some examples, the first suture lines 414 can be positioned such that they are aligned with corners of the struts 240 between the first face 424 and the fourth face 434 of the respective strut, and are adjacent to the wedge member 446. In some examples, first securing loops 450 (that is, wedge securing loops 450) can each extend from one of the first suture lines 414 and through the wedge member 446. When the first securing loops 450 are tightened, the commissure wedge member 446 can be pressed radially inwards against the commissure tabs 408, compressively securing the commissure tabs 408 between the wedge member 446 and the faces 434, 424 of the struts 240. In some examples, a single securing loop can extend to both of the first suture lines and through the wedge member 446 (similar to the securing loop 350 in FIG. 7).

The securing loops 450 can each pass through at least one stitch 418 in one of the suture lines 414. In some examples, the securing loops 450 can each extend only through a single stitch 418 of each of the suture lines 414. In some examples, the securing loops 450 can each extend through multiple stitches 418 of the suture lines 414, such that the securing loops 450 forms multiple loops extending continuously back and forth between the suture line 414 and the wedge member 446 along the length of the respective suture line 414 in a helical or zig-zag pattern. In some examples, a plurality of discrete securing loops 450 can be formed, each of which extends through a respective stitch 418 and the wedge member 446 along the length of the suture lines 414.

In some examples, portions of the reinforcing members 410a (on the interior of the frame in FIG. 9) can be wrapped around the window struts 240. As shown in FIG. 10, each of the reinforcing members 410a can extend over and be positioned on second and third faces 430, 432 of the respective strut 240. Each of the reinforcing members 410a can further extend over the face 434 of the respective strut 240 such that the reinforcing members 410a can cover an exterior surface of the commissure tabs 408. In this configuration, side edges 458 (opposing the side edges 452) of the reinforcing members 410a can be positioned adjacent or near each other exterior of the frame 202. Thus, in some examples, portions of the commissure tabs 408 that are exterior of the commissure window 242 can be sandwiched between two layers of the reinforcing members 410a. In this position, the second suture lines 422 of the reinforcing members 410 can be positioned outside of the commissure window 242 (that is, outside of the frame 202), and a securing loop 454 (also referred to as “an outer securing loop”) can extend between the suture lines 422 on the reinforcing members 410a to secure the adjacent ends of the reinforcing members to each other. In some examples, two securing loops 454 can be included that each extend from one of the suture lines 422 to the wedge member 446 and/or to the securing loops 450 (rather than extending between the opposing suture lines 422 on the reinforcing members 410a).

The securing loop 454 can pass through at least one stitch 418 of each of the suture lines 422. In some examples, the securing loop 454 can extend only through a single stitch 418 of each of the suture lines 422. In some examples, the securing loop 454 can extend through multiple stitches 418 of the suture lines 422, such that the securing loop 454 forms multiple loops extending continuously back and forth between the suture lines 422 on the reinforcing members 410a along the length of the suture lines 422 in a helical or zig-zag pattern. In some examples, a plurality of discrete securing loops 454 can be formed, each of which extends through respective pairs of stitches 418 in the suture lines 422 along the length of the suture lines 422. In some examples, the securing loop 454 can additionally extend through the wedge member 446.

When the securing loop 454 is tightened, the securing members 410a are fixed on the exterior of the commissure and resist and/or prevent movement of the securing members 410a relative to commissure window 242. Additionally, the commissure wedge member 446 can be further pressed radially inwards against the commissure tabs 408 for securing the commissure tabs 408 between the wedge member 446 and the faces 434 of the struts 240.

Another exemplary commissure configuration that can be formed using the leaflet 400 is shown in FIG. 11. As discussed above, different from the reinforcing members 410a, the side edges 452 the reinforcing members 410b can extend past the side edges 438 of the commissure tabs 408. In some examples, the reinforcing members 410b can have greater length than the reinforcing members 410a, which can enable folding at the opposing ends of the reinforcing members 410b, as described below.

In some examples, a first stage of forming a commissure using adjacent leaflets 400 including the securing members 410b can be similar to the example described above for the leaflets 400 including the securing members 410a.

In some examples, after insertion of the commissure tabs 408 and the reinforcing members 410b through the commissure window 242, the side edges 452 of each reinforcing member 410b can be folded inwardly (toward the leaflet body 402) to form a first folded portion 460 at one end of each of the reinforcing member 410b. In some examples, the side edge 452 can remain unfolded, and the folded portions 460 can be excluded from the commissure configuration.

Portions of the commissure tabs 408 and the reinforcing members 410b that are exterior of the frame 202 (including the folded portions 460) can be wrapped over the struts 240 to extend over the fourth and third faces 434, 432 thereof. In some examples, the reinforcing members 410b can be positioned on the fourth and third faces 434, 432 and can be disposed between the commissure tabs 408 and the fourth and third faces 434, 432 of the respective strut 240. Additionally, the folded portions 460 (if included) can extend over and align with the third faces 432 of the struts 240.

Similar to the manner described above with reference to FIG. 9, a commissure wedge member 446 can be positioned between the adjacent commissure tabs 408 at a location where they exit the commissure window 242, and first securing loops 450 (or a single first securing loop) can attach the wedge member 446 to each of the first suture lines 414.

In some examples, side edges 458 (opposing the side edges 452) of the reinforcing members 410b can be folded outwardly (away from the leaflet body 402) to form second folded portions 462. In some examples, the side edge 458 can remain unfolded, and the folded portion 462 can be excluded from the commissure configuration.

Portions of the reinforcing members 410b on the interior of the frame (for example, portions similar to the portions of the reinforcing members 410a on the interior of the frame 202 in FIG. 9) can be wrapped over the window struts 240 so that they extend around second and third faces 430, 432 of the respective strut 240. The folded portions 462 (if included) can extend over and align with the third faces 432 of the struts 240.

As shown in FIG. 11, the second suture lines 422 of the reinforcing members 410b can be positioned such that they are disposed on opposing sides of the commissure formed in the commissure window 242. In some examples, exterior surfaces of the commissure tabs 408 (exterior of the frame 202) are not covered by the reinforcing members 410b. In some examples, end portions of the commissure tabs 408 that extend over the third faces 432 of the struts 240 can be sandwiched between the first and second folded portions 460, 462 (that is, between four layers of the reinforcing members 410b).

In some examples, a securing loop 456 (also referred to as “an outer securing loop”) can extend between the suture lines 422 on the reinforcing members 410b, and can extend over the exterior surface of the commissure tabs 408. In some examples, two securing loops 456 can be included that each extend from one of the suture lines 422 to the wedge member 446 and/or to the securing loops 450 (rather than extending between the opposing suture lines 422 on the reinforcing members 410b).

The securing loop 456 can pass through at least one stitch 418 of each of the suture lines 422 in the reinforcing members 410b. In some examples, the securing loop 456 can extend only through a single stitch 418 of each of the suture lines 422. In some examples, the securing loop 456 can extend through multiple stitches 418 of the suture lines 422, such that the securing loop 456 forms multiple loops extending continuously back and forth between the suture lines 422 on the reinforcing members 410b along the length of the suture lines 422 in a helical or zig-zag pattern. In some examples, a plurality of discrete securing loops 456 can be formed, each of which extends through respective pairs of stitches 418 in the suture lines 422 along the length of the suture lines 422. In some examples, the securing loop 456 can additionally extend through the wedge member 446.

When the securing loop 456 is tightened, the securing members 410b can be fixed at sides of the commissure window 242 and resist and/or prevent movement of the securing members 410b relative to commissure window 242. Additionally, the commissure wedge member 446 can be further pressed radially inwards against the commissure tabs 408, further compressively securing the commissure tabs 408 between the wedge member 446 and the four faces 434 of the struts 240.

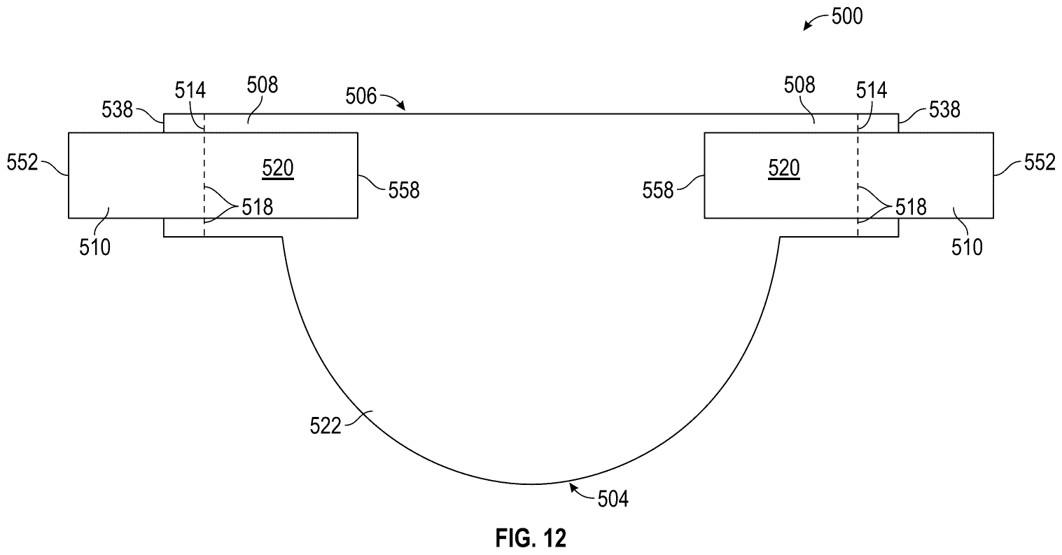

FIG. 12 illustrates a front elevation view of another exemplary leaflet 500 including attached reinforcing members 510. In some examples, the leaflet 500 can have similar characteristics with and may be used in the same applications as the leaflets 204, 300, 400, described above. For example, the leaflet 500 can include a leaf-shaped leaflet body 502 with an inflow end portion (or edge) 504 and an outflow end portion (or edge) 506 and opposing commissure tabs 508 for attachment to a commissure window of a frame. The reinforcing members 510 can each be attached to one of the commissure tabs 508. The commissure tabs 508 can be paired with commissure tabs of adjacent leaflets, thereby forming a valvular structure (which can be similar to, for example, the valvular structure 14 of FIG. 1 and/or the valvular structure formed from the leaflets 204 shown in FIG. 3).

As shown in FIG. 12, in some examples, the reinforcing member 510 can cover a portion of the respective commissure tab 508. In some examples, the reinforcing members 510 can cover the entirely of the respective commissure tab 508. In some examples, the reinforcing members 510 can be centered relative to the respective commissure tabs 508 in an axial direction. In some examples, the reinforcing members 510 can be offset in an axial direction relative to the respective commissure tabs 508 (similar to the reinforcing member reinforcing member 310 and the commissure 308 shown in FIG. 5).

In some examples, the reinforcing members 510 can be made of any of various biocompatible fabrics, such as polyethylene terephthalate (PET) fabric, or non-fabric materials, such as polyurethane, silicone, or other polymers. In some examples, the reinforcing members 510 can be made of natural tissue, such as pericardium. In some examples, the reinforcing members 510 can comprises a combination of natural tissue and a synthetic material.

Each of the reinforcing members 510 can be attached to the leaflet 500 by a single suture line (also referred to as a stitch line). As shown in FIG. 12, a suture line 514 can extend through the reinforcing member 510 and the commissure tab 508, thereby securing the reinforcing members 510 to the respective commissure tabs 508. In some examples, the suture lines 514 can be similar to the first suture lines 314, 414. For example, the suture lines 514 can each form a plurality of in-and-out stitches 518 that extend out of a face 520 of the respective reinforcing member 510, as well as out of an opposite face of the respective commissure tab 508. Different from the leaflet 300 and the reinforcing member 310, in some examples, only one suture line (the suture line 514) extends through the reinforcing member 510 and the commissure tab 508. Different from the leaflets 300, 400 and the reinforcing members 310, 410, the reinforcing member 510 lacks any additional sutures that extend solely through the reinforcing member. In other words, suture lines similar to the suture line 316 and the suture lines 322, 422 are excluded from the leaflet 500. Thus, the suture line 514 can provide a sole attachment structure for connecting sutures (or securing loops).

In some examples, as described above for the suture lines 314, 414, the locations of the suture lines 514 on the reinforcing members 510 can be selected based geometries and/or sizes of the struts 240, the commissure window 242, the commissure tabs 508, and/or a desired commissure configuration. In some examples, a length of the reinforcing member 510 and/or a position of the reinforcing members 510 relative to the commissure tabs 508 can be selected based geometries and/or sizes of the struts 240, the commissure window 242, the commissure tabs 508, and/or a desired commissure configuration. In some examples, a side edge 552 of the reinforcing member 510 can extend past a side edge 538 of the commissure tab 508, as illustrated in FIG. 12. In some examples, the side edge 538 of the commissure tab 508 can extend past the side edge 552 of the reinforcing member 510.

The arrangement of the commissure tabs 508, the reinforcing members 510, and the suture line 514 can allow the leaflets 500 to be attached to a commissure window (for example, commissure window 20 of frame 12 or commissure 242 of frame 202) with minimal need for additional leaflet material to reinforce attachment points, while avoiding load-bearing stitches that extend through the leaflet or at least minimizing load-bearing on stitches that extend through the leaflet, as described in greater detail herein. Further, the reduced number of suture lines relative to the leaflets 300, 400 (including the reinforcing members 310, 410) simplifies assembly of the leaflet 500, while still preventing and/or minimizing load-bearing on stitches that extend through the leaflet.

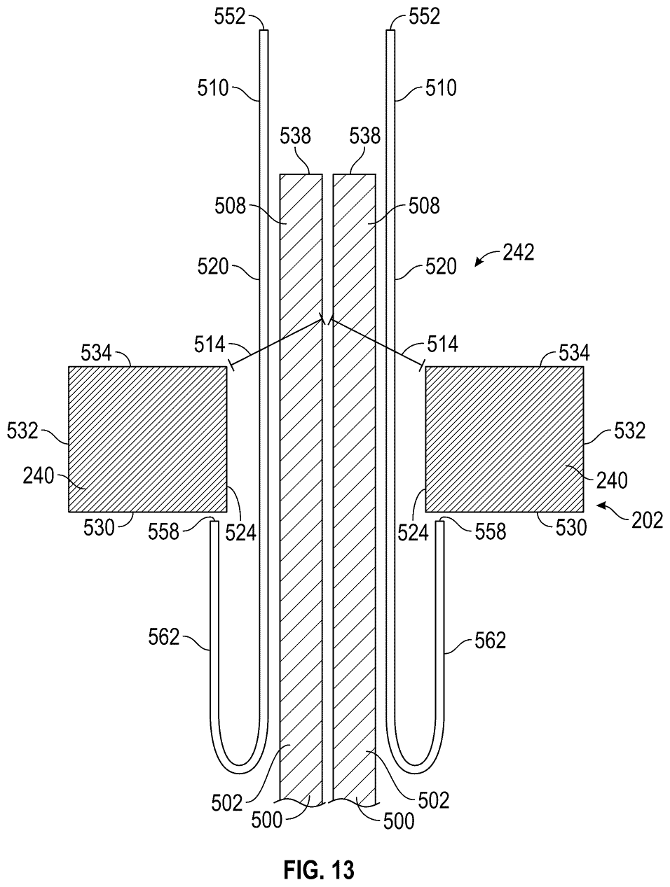

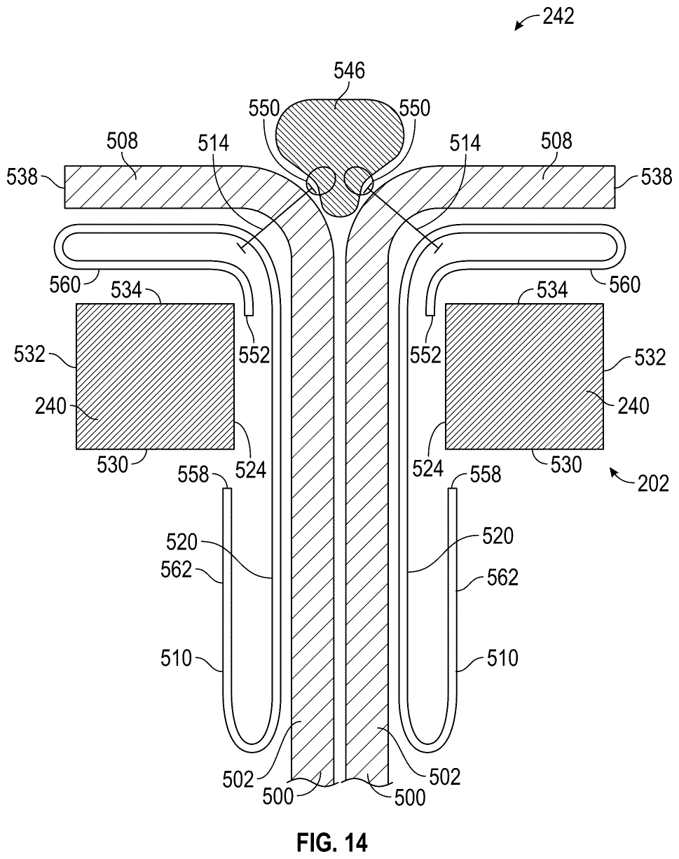

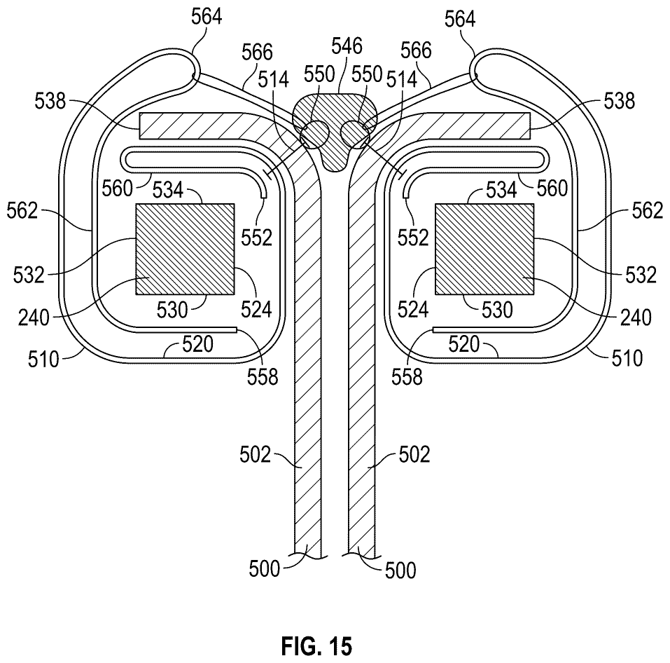

An exemplary first stage of attachment of paired commissure tabs 508 of adjacent leaflets 500 to window struts 240 of a commissure window 242 is illustrated in FIG. 13. An exemplary second stage of attachment of the paired commissure tabs 508 of adjacent leaflets 500 to the struts 240 of the commissure window 242 is illustrated in FIG. 14. An exemplary third stage of attachment of the paired commissure tabs 508 of adjacent leaflets 500 to the struts 240 of the commissure window 242 is illustrated in FIG. 15, and shows an exemplary configuration for a commissure that can be formed with the leaflets 500 including the reinforcing members 510. It will be appreciated that FIGS. 13-15 are not drawn to scale to improve clarity in the depiction of the interaction between the components and/or the arrangement of the components, such as the leaflet 500, the reinforcing members 510, the suture lines 514, the window struts 240 of the commissure window 242, and the connecting sutures (discussed below).

To attach the commissure tabs 508 of the leaflets 500 and the reinforcing members 510 to a window strut 240 of a commissure window 242, the commissure tabs 508 and the reinforcing members 510 can be inserted through the commissure window 242 from inside the frame 202 and can extend radially outward past the window strut 240 such that a portion of each commissure tab 508 and reinforcing member 510 is outside the frame 202. As shown in FIG. 13, at the first stage of formation, the suture lines 514 can be positioned radially outwards of the window strut 240 or the commissure window 242 (outside of the frame 202). The reinforcing members 510 can be positioned against a first face 524 of each of the window struts 240. Side edges 558 of the reinforcing members 510 can be folded outward (away from the leaflet body 502) to create folded portions 562 in the reinforcing members 510 in portions thereof that are interior of the frame 202.

In the second stage of attachment shown in FIG. 14, a commissure wedge member 546 can be positioned between the adjacent commissure tabs 508 at a location where they exit the commissure window 242, and first securing loops 550 can attach the wedge member 546 to each of the suture lines 514 (in a manner similar to attachment of the wedge member 446 to the suture lines 414 described above with reference to FIG. 9).

In some examples, side edges 552 (opposing the side edges 558) of the reinforcing members 510 can be folded outwardly (away from the leaflet body 502) to form second folded portions 560 exterior of the frame 202. In some examples, the side edges 552 can remain unfolded, and the folded portions 560 can be excluded from the commissure configuration.

After insertion of the commissure tabs 508 and the reinforcing members 510 through the commissure window 242 (and formation of the folded portions 560, if included), the commissure tabs 508 and the reinforcing members 510 can be wrapped over the struts 240 to extend over fourth faces 534 thereof. In some examples, the folded portions 560 of the reinforcing members 510 can be positioned on the fourth faces 534 and can be disposed between the commissure tabs 508 and the fourth face 534 of the respective strut 240. In some examples, the bends in the commissure tabs 508 can be right angle bends or approximately right-angle bends (that is, the bends can each be 90-degree bends or approximately 90-degree bends).

As shown in FIG. 15, portions of the reinforcing members 510 on the interior of the frame 202 (such as, portions of the reinforcing members 510 on the interior of the frame 202 in FIGS. 13 and 14) can be wrapped over the window struts 240 so that they extend around second and third faces 530, 532 and over at least a portion of the fourth faces 534 of the respective strut 240. In some examples, the side edges 558 can be aligned with corners at intersections between the first and second faces 524, 530 of the struts 240, such that the folded portions 562 wrap around the struts and closed ends 564 of the folded portions 562 are exterior of the frame 202. In some examples, at least a portion of the exterior surfaces of the commissure tabs 508 (exterior of the frame 202) can be covered by the reinforcing members 510. In some examples, portions of the commissure tabs 508 that extend over the fourth faces 534 of the struts 240 can be sandwiched between the first and second folded portions 560, 562 (that is, between four layers of the reinforcing members 510).

In some examples, securing loops 566 (also referred to as “an outer securing loops”) can extend between the closed ends 564 of the folded portions 562 and the securing loops 550. In some examples, a single securing loop 566 can extend between the closed ends 564 of the folded portions (rather two loops).

The securing loops 566 can pass through at least one stitch of each of the securing loops 550. In some examples, securing loops 566 can pass through an upper stitch and a lower stitch of the securing loops 550 that correspond to a height of the securing member 510. In some examples, the securing loops 566 can additionally or alternatively extend through the wedge member 546.

When the securing loops 566 are tightened, the securing members 510 can be fixed at the exterior side of the commissure window 242 and resist and/or prevent movement of the securing members 510 relative to commissure window 242. Additionally, the commissure wedge member 546 can be further pressed radially inwards against the commissure tabs 508, further compressively securing the commissure tabs 508 between the wedge member 546 and the fourth faces 534 of the struts 240.

As discussed above with respect to the leaflet 300, in some examples, a prosthetic heart valve, such as the prosthetic heart valve 10 and the prosthetic heart valve 200, comprises multiple commissure windows 242 for multiple leaflets 400, 500, forming a corresponding number of commissures assembled in the manners described above in FIGS. 8-15. For example, a prosthetic heart valve can have two or more leaflets 400, 500, such as three leaflets 400, 500, or more than three leaflets 400, 500, wherein each leaflet 400, 500 comprises two commissure tabs 408, 508, and a matching number of commissure windows 242 receive paired commissure tabs of adjacent leaflets to form a number of commissures equal to the number of leaflets, such as two commissures, three commissures, or more than three commissures.

While the examples described herein pertain to a commissure window formed by two rectangular window struts, such as window struts, it is understood that the geometry of the window struts can be varied according to the needs of the frame. For example, the window struts can have a circular, ovular, or polygonal cross section. In such examples, the reinforcing members 410, 510 and the commissure tabs 408, 508 can be wrapped or folded around the circumference, perimeter, or any number of sides of the window struts.

Similar to the example commissures described above with respect to FIGS. 6A-7, the commissure configurations of FIGS. 9-11 and 13-15 can form one or more relatively narrow commissure assemblies that are nonetheless durable can be assembled. Because the tensioning forces securing the commissure tabs 408, 508 to the window strut are borne at least by the various securing loops (such as the securing loops 450, 454, 456, 550, 566. Accordingly, no or fewer stress concentrators are added to the leaflet, thereby improving leaflet durability.

Also, in some examples, because no suture line attaches any leaflet to itself or to another leaflet, each of the window struts 240 needs only accommodate a single layer of leaflet material (that is, a single commissure tab 408, 508 folded around the window strut 240), the assembly comprising the commissure window 242, the commissure tabs 408, 508, and the reinforcing members 410, 510 as described herein, for example, may have a reduced profile compared to known prosthetic valves. This, in turn, reduces the overall crimp profile of the prosthetic heart valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

Example 1. A prosthetic heart valve comprising a radially expandable frame having a first end portion, a second end portion and a longitudinal axis extending between the first end portion and the second end portion, and a plurality of commissure windows; and a valvular structure disposed within and attached to the radially expandable frame, the valvular structure comprising at least a first leaflet having a first commissure tab and a first reinforcing member attached to the first commissure tab and a second leaflet having a second commissure tab and a second reinforcing member attached to the second commissure tab; wherein the first and second commissure tabs are positioned adjacent each other and extend through a first commissure window of the plurality of commissure windows, the first commissure window being defined by first and second axially extending posts, wherein the first commissure tab forms two or more bends around the first axially extending post and the second commissure tab forms two or more bends around the second axially extending post.

Example 2. The prosthetic heart valve of any example herein, particularly example 1, wherein each of the first and second leaflets comprise an outflow edge portion that is movable towards and away from the radially expandable frame under the flow of blood.

Example 3. The prosthetic heart valve of any example herein, particularly examples 1-2, wherein the first reinforcing member is attached to the first commissure tab by a first suture line extending through the first leaflet and the first reinforcing member and a second suture line extending through the first leaflet and the first reinforcing member, and wherein the second suture line is spaced apart from the first suture line along a length of the first reinforcing member.

Example 4. The prosthetic heart valve of any example herein, particularly example 3, wherein the first suture line and the second suture line are positioned radially outwards of the first commissure window.

Example 5. The prosthetic heart valve of any example herein, particularly examples 3-4, further comprising a third suture line extending through the first reinforcing member and spaced apart from the first suture line and the second suture line along a length of the first reinforcing member.

Example 6. The prosthetic heart valve of any example herein, particularly example 5, wherein the third suture line is positioned radially inwards of the first commissure window.

Example 7. The prosthetic heart valve of any example herein, particularly example 6, further comprising a first securing loop extending between the second suture line and the third suture line.

Example 8. The prosthetic heart valve of any example herein, particularly example 7, wherein the first securing loop extends around the first axially extending post, and wherein when the first securing loop is tightened, the first reinforcing member is folded to at least partially enclose the first axially extending post.

Example 9. The prosthetic heart valve of any example herein, particularly examples 7-8, further comprising a second securing loop extending between the first suture line and the third suture line.

Example 10. The prosthetic heart valve of any example herein, particularly example 9, wherein the second securing loop extends around an end portion of the first leaflet, and wherein when the second securing loop is tightened, the end portion of the first leaflet is folded to at least partially enclose the first axially extending post and the first reinforcing member.

Example 11. The prosthetic heart valve of any example herein, particularly examples 1-10, wherein the valvular structure further comprises a third leaflet and a third reinforcing member, and wherein the radially expandable frame comprises at least three commissure windows.

Example 12. The prosthetic heart valve of claim 11, wherein the first leaflet is not sutured to the third leaflet.

Example 13. The prosthetic heart valve of any of claims 3-12, wherein the first leaflet is not sutured to itself, the second leaflet, or the third leaflet.

Example 14. The prosthetic heart valve of any example herein, particularly examples 9-13, wherein at least one of the first suture line, the second suture line, or the third suture line comprises a plurality of in-and-out stitches.

Example 15. The prosthetic heart valve of any example herein, particularly example 14, wherein the first securing loop or the second securing loop passes through two or more stitches of the first suture line, the second suture line, or the third suture line.

Example 16. The prosthetic heart valve of any example herein, particularly examples 1-16, wherein the prosthetic heart valve further comprises an outer sealing member positioned radially outwards of the radially expandable frame.

Example 17. The prosthetic heart valve of any example herein, particularly examples 1-16, further comprising a wedge member, wherein the wedge member is disposed radially outwards of the first commissure window and configured to urge at least the first commissure tab against the first axially extending post.

Example 18. The prosthetic heart valve of any example herein, particularly example 17, wherein the wedge member secures the first and second commissure tabs against respective sides of the first commissure window, and wherein a securing loop extends between a corresponding suture line of the first and second leaflets and around the wedge member.

Example 19. A commissure assembly for a prosthetic heart valve, the prosthetic heart valve comprising a radially expandable frame with a commissure window having a first axially extending post and a second axially extending post, the commissure assembly comprising a at least a first leaflet having a first commissure tab and a second leaflet having a second commissure tab; a first reinforcing member sutured to the first commissure tab and a second reinforcing member sutured to the second commissure tab; a first suture line and a second suture line extending through the first reinforcing member and the first leaflet; a third suture line extending through the first reinforcing member; a first securing loop extending between the second suture line and the third suture line; and a second securing loop extending between the first suture line and the third suture line; wherein the first commissure tab and the second commissure tab extend through the commissure window to form a commissure, wherein the first securing loop extends around the first axially extending post of the commissure window, and the second securing loop extends around an end portion of the first leaflet, and wherein the first commissure tab is folded to at least partially enclose the first axially extending post.

Example 20. The commissure assembly of any example herein, particularly example 19, wherein the first axially extending post has a first face, a second face, and third face, and a fourth face, and wherein the first reinforcing member is folded to surround each of the four faces of the first axially extending post.

Example 21. The commissure assembly of any example herein, particularly example 19, wherein the first reinforcing member encircles the first axially extending post.

Example 22. The commissure assembly of any example herein, particularly examples 19-21, wherein when the first securing loop is tightened, the first reinforcing member is folded or wrapped around the first axially extending post.

Example 23. The commissure assembly of any example herein, particularly examples 19-22, wherein when the second securing loop is tightened, the first commissure tab is folded or wrapped around the first axially extending post.

Example 24. The commissure assembly of any example herein, particularly example 19-23, further comprising a wedge member that extends between the first commissure tab and the second commissure tab.

Example 25. The commissure assembly of any example herein, particularly example 24, further comprising a wedge securing loop extending between the second suture line of the first commissure tab and a suture line of the second commissure tab, wherein the wedge securing loop extends radially outwards of the wedge member.

Example 26. The commissure assembly of any example herein, particularly examples 19-25, wherein any of the first suture line, the second suture line, or the third suture line comprises a plurality of in-and-out stitches extending in a longitudinal direction.

Example 27. The commissure assembly of any example herein, particularly example 26, wherein either of the first securing loop or the second securing loop passes through two or more stitches of any of the first, second or third suture lines.

Example 28. The commissure assembly of any example herein, particularly examples 19-27, wherein no suture line attaches any leaflet to itself or to another leaflet.

Example 29. The commissure assembly of any example herein, particularly examples 19-28, wherein the first reinforcing member separates the first leaflet from the first axially extending post and the second reinforcing member separates the second leaflet from the second axially extending post.

Example 30. The commissure assembly of any example herein, particularly examples 19-29, further comprising an outer sealing member positioned radially outwards of the radially expandable frame.

Example 31. A prosthetic heart valve, comprising: a radially expandable frame having a first end portion, a second end portion, and a longitudinal axis extending between the first end portion and the second end portion, and a plurality of commissure windows; and a valvular structure disposed within and attached to the radially expandable frame, the valvular structure comprising at least a first leaflet having a first commissure tab and a first reinforcing member attached to the first commissure tab and a second leaflet having a second commissure tab and a second reinforcing member attached to the second commissure tab; wherein the first and second commissure tabs are positioned adjacent each other and extend through a first commissure window of the plurality of commissure windows, the first commissure window being defined by first and second axially extending posts, wherein the first reinforcing member is attached to the first commissure tab by a first suture line; and wherein no suture line attaches any leaflet to itself or to another leaflet.

Example 32. The prosthetic heart valve of any example herein, particularly example 31, further comprising a second suture line, and a third suture line, wherein the second suture line is spaced apart from the first suture line along a length of the first reinforcing member and the third suture line is spaced apart from the second suture line along the length of the first reinforcing member.

Example 33. The prosthetic heart valve of any example herein, particularly example 32, wherein the first suture line and the second suture line pass through the first reinforcing member and the first commissure tab.

Example 34. The prosthetic heart valve of any example herein, particularly examples 32-33, wherein the third suture line passes only through the first reinforcing member.

Example 35. The prosthetic heart valve of any example herein, particularly examples 32-34 wherein the first suture line and the second suture line are positioned radially outwards of the first commissure window and the third suture line is positioned radially inwards of the first commissure window.

Example 36. The prosthetic heart valve of any example herein, particularly examples 32-35, further comprising a first securing loop extending from the second suture line to the third suture line.

Example 37. The prosthetic heart valve of any example herein, particularly example 36, wherein the first securing loop extends around the first axially extending post.

Example 38. The prosthetic heart valve of any example herein, particularly example 37, wherein when the first securing loop is tightened, the first reinforcing member is wrapped around the first axially extending post.

Example 39. The prosthetic heart valve of any example herein, particularly examples 36-38, wherein the valvular structure further comprises a second securing loop extending from the first suture line to the third suture line.

Example 40. The prosthetic heart valve of any example herein, particularly example 39, wherein the second securing loop extends around the first commissure tab.

Example 41. The prosthetic heart valve of any example herein, particularly example 40, wherein when the second securing loop is tightened, the first commissure tab is wrapped around the first axially extending post.

Example 42. The prosthetic heart valve of any example herein, particularly examples 39-41, wherein each of the first suture line, the second suture line, and the third suture line defines a plurality of in-and-out stitches.

Example 43. The prosthetic heart valve of any example herein, particularly example 42, wherein the first securing loop or the second securing loop passes through at least two stitches of two of the first, second, and third suture lines.

Example 44. The prosthetic heart valve of any example herein, particularly examples 31-43, wherein the first reinforcing member separates the first leaflet from the first axially extending post and the second reinforcing member separates the second leaflet from the second axially extending post.

Example 45. The prosthetic heart valve of any example herein, particularly examples 31-44, wherein the first reinforcing member extends completely around the first axially extending post and the second reinforcing member extends completely around the second axially extending post.

Example 46. The prosthetic heart valve of any example herein, particularly examples 31-45, further comprising an outer sealing member positioned radially outwards of the radially expandable frame.

Example 47. A prosthetic heart valve, comprising: a radially expandable frame having a first end portion, a second end portion, and a longitudinal axis extending between the first end portion and the second end portion, and a plurality of commissure windows; a plurality of leaflets disposed within and attached to the radially expandable frame and forming a valvular structure configured to regulate a flow of blood through the radially expandable frame; and wherein a first leaflet of the plurality of leaflets comprises a first commissure tab and a first reinforcing member is attached to the first commissure tab, wherein the first commissure tab extends through a first commissure window of the plurality of commissure windows defined by a first axially extending post and a second axially extending post, and wherein the first axially extending post has a rectangular cross section and the first commissure tab extends parallel to a first face, a second face, and a third face of the first axially extending post when secured.

Example 48. The prosthetic heart valve of any example herein, particularly example 47, wherein the first reinforcing member is positioned between the first axially extending post and the first commissure tab.

Example 49. The prosthetic heart valve of any example herein, particularly examples 46-48, wherein the first reinforcing member is attached to the first leaflet by a plurality of suture lines.

Example 50. The prosthetic heart valve of any example herein, particularly example 49, wherein the plurality of suture lines comprises a first suture line and a second suture line spaced apart from the first suture line along the first commissure tab and wherein the prosthetic heart valve further comprises a third suture line spaced apart from the first suture line and the second suture line along the first reinforcing member.

Example 51. The prosthetic heart valve of any example herein, particularly example 50, wherein the first suture line and the second suture line are positioned radially outwards from the first commissure window and the third suture line is positioned radially inwards from the first commissure window.

Example 52. The prosthetic heart valve of any example herein, particularly example 51, wherein the first suture line and the second suture line extend through the first leaflet and the first reinforcing member, and wherein the third suture line extends through the first reinforcing member.

Example 53. The prosthetic heart valve of any example herein, particularly examples 50-52, further comprising a first securing loop extending from the second suture line to the third suture line.

Example 54. The prosthetic heart valve of any example herein, particularly example 53, wherein when the first securing loop is tightened, the first reinforcing member is folded around the first axially extending post.

Example 55. The prosthetic heart valve of any example herein, particularly examples 53-54, further comprising a second securing loop extending from the first suture line to the third suture line.

Example 56. The prosthetic heart valve of any example herein, particularly example 55, wherein when the second securing loop is tightened, the first commissure tab of the first leaflet is folded around the first axially extending post.

Example 57. The prosthetic heart valve of any example herein, particularly examples 55-56, wherein each of the first suture line, the second suture line, and the third suture line defines a plurality of in-and-out-stitches.

Example 58. The prosthetic heart valve of any example herein, particularly example 57, wherein one of the first securing loop or the second securing loop passes through at least two stitches of two of the first, second, and third suture lines.

Example 59. The prosthetic heart valve of any example herein, particularly examples 47-58, further comprising a wedge member extending between the first commissure tab, and wherein a securing loop positioned radially outwards of the wedge member extends between a suture line in the first leaflet and a suture line in a second commissure tab.

Example 60. The prosthetic heart valve of any example herein, particularly examples 47-59, further comprising an outer sealing member positioned radially outwards from and secured to the radially expandable frame.

Example 61. A prosthetic heart valve, comprising: a radially expandable frame having a first end portion, a second end portion, and a longitudinal axis extending between the first end portion and the second end portion, and a plurality of commissure windows; a plurality of leaflets disposed within and attached to the radially expandable frame and forming a valvular structure configured to regulate a flow of blood through the radially expandable frame; and a plurality of reinforcing members attached to the plurality of leaflets; wherein a first leaflet of the plurality of leaflets comprises a first commissure tab and a first reinforcing member is attached to the first commissure tab, wherein the first commissure tab extends through a first commissure window of the plurality of commissure windows defined by a first axially extending post and a second axially extending post, and wherein the first axially extending post has a rectangular cross section, and wherein the first reinforcing member encloses the first axially extending post.

Example 62. The prosthetic heart valve of any example herein, particularly example 61, wherein the first reinforcing member is positioned between the first axially extending post and the first commissure tab.

Example 63. The prosthetic heart valve of any example herein, particularly examples 61-62, wherein the first reinforcing member is attached to the first leaflet by a plurality of suture lines.

Example 64. The prosthetic heart valve of any example herein, particularly example 63, wherein the plurality of suture lines comprises a first suture line and a second suture line spaced apart from the first suture line along the first commissure tab, and the prosthetic heart valve further comprises a third suture line spaced apart from the first suture line and the second suture line along the first reinforcing member.

Example 65. The prosthetic heart valve of any example herein, particularly example 64, wherein the first suture line and the second suture line are positioned radially outwards from the first commissure window and the third suture line is positioned radially inwards from the first commissure window.

Example 66. The prosthetic heart valve of any example herein, particularly example 65, wherein the first suture line and the second suture line extend through the first commissure tab and the first reinforcing member, and wherein the third suture line extends through the first reinforcing member.

Example 67. The prosthetic heart valve of any example herein, particularly examples 61-66, further comprising a first securing loop extending from the second suture line to the third suture line.

Example 68. The prosthetic heart valve of any example herein, particularly example 67, wherein when the first securing loop is tightened, the first reinforcing member is folded around the first axially extending post.

Example 69. The prosthetic heart valve of any example herein, particularly examples 61-68, further comprising a second securing loop extending from the first suture line to the third suture line.

Example 70. The prosthetic heart valve of any example herein, particularly example 69, wherein when the second securing loop is tightened, the first commissure tab is folded around the first axially extending post.

Example 71. A method of attaching a leaflet to a prosthetic heart valve, comprising: attaching a reinforcing member to a leaflet with a first suture line and a second suture line to form a leaflet assembly, the second suture line being spaced apart from the first suture line along a commissure tab of the leaflet, the first suture line and the second suture line extending through the reinforcing member and the leaflet; attaching a third suture line to the reinforcing member, spaced apart from the second suture line along a length of the reinforcing member; arranging the leaflet assembly inside a radially expandable frame comprising a commissure window defined by a first axially extending window strut and a second axially extending window strut, such that an end portion of the leaflet assembly extends through the commissure window with the third suture line being positioned radially inwards of the commissure window and the first suture line and the second suture line being positioned radially outwards from the commissure window; folding a first end portion of the reinforcing member around the first axially extending window strut; and folding the end portion of the leaflet at least partially around the first axially extending window strut and the reinforcing member.

Example 72. The method of any example herein, particularly example 71, wherein folding the first end portion of the reinforcing member at least partially around the first axially extending window strut comprises attaching a securing loop to the third suture line and to the second suture line, such that the securing loop extends around the first axially extending window strut.

Example 73. The method of any example herein, particularly example 72, further comprising tightening the securing loop to fold the reinforcing member around the first axially extending window strut.

Example 74. The method of any example herein, particularly examples 72-73, wherein the securing loop is a first securing loop, further wherein folding the end portion of the leaflet at least partially around the first axially extending window strut and the reinforcing member comprises attaching a second securing loop to the third suture line and the first suture line, such that the second securing loop extends around the end portion of the leaflet.

Example 75. The method of any example herein, particularly example 74, further comprising tightening the second securing loop to fold the end portion of the leaflet at least partially around the first axially extending window strut and the reinforcing member.

Example 76. The method of any example herein, particularly example 75, wherein each of the first suture line, the second suture line, and the third suture line comprises a plurality of in-and-out stitches.

Example 77. The method of any example herein, particularly example 76, wherein the first securing loop extends through at least one stitch of the second suture line and at least one stitch of the third suture line, and the second securing loop extends through at least one stitch of the first suture line and at least one stitch of the third suture line.

Example 78. The method of any example herein, particularly examples 71-77, wherein no leaflet is sutured to any other leaflet or to itself.

Example 79. The method of any example herein, particularly examples 71-78, wherein the leaflet assembly is a first leaflet assembly and the method further comprises attaching a second leaflet assembly to the radially expandable frame, such that the second leaflet assembly and the first leaflet assembly define a commissure, and the end portion of the first leaflet assembly and an end portion of the second leaflet assembly extend through the commissure window.

Example 80. The method of any example herein, particularly example 79, further comprising attaching one or more additional leaflet assemblies to the radially expandable frame to form one or more additional commissures, each commissure extending through a corresponding commissure window.

Example 81. The method of any example herein, particularly example 79, further comprising attaching a wedge member to the commissure, wherein the wedge member is positioned radially outwards of the commissure window and extends between the leaflet assemblies of the commissure and wherein the wedge member is attached to the first and second leaflet assemblies by a securing loop extending between the second suture line of the first leaflet and the second suture line of the second leaflet.

Example 82. A method of assembling a prosthetic heart valve, comprising:

assembling a first leaflet assembly by suturing a first reinforcing member to a first leaflet with first and second suture lines extending through the first leaflet and the first reinforcing member; forming a third suture line along the first reinforcing member; inserting a first commissure tab of the first leaflet assembly through a first commissure window of a frame of the prosthetic heart valve; forming a first securing loop that extends through the second and third suture lines and around a first axially extending window strut of the first commissure window; and tightening the first securing loop to fold the first reinforcing member and the first commissure tab around the first axially extending window strut.

Example 83. The method of any example herein, particularly example 82, wherein when the first commissure tab is inserted into the first commissure window, the first suture line and the second suture line are positioned radially outwards of the first commissure window and the third suture line is positioned radially inwards of the first commissure window.

Example 84. The method of any example herein, particularly examples 82-83, further comprising forming a second securing loop that extends through the third suture line and the first suture line, such that the second securing loop extends around an end portion of the first leaflet assembly, and tightening the second securing loop to fold the end portion of the first leaflet assembly around the first axially extending window strut.

Example 85. The method of any example herein, particularly example 84, wherein each the first suture line, the second suture line, and the third suture line comprises a plurality of in-and-out-stitches.

Example 86. The method of any example herein, particularly example 85, wherein the first securing loop extends through at least one stitch of the second suture line and at least one stitch the third suture line.

Example 87. The method of any example herein, particularly example 86, wherein the second securing loop extends through at least one stitch of the first suture line and at least one stitch of the third suture line.

Example 88. The method of any example herein, particularly examples 82-87, further comprising: assembling a second leaflet assembly by suturing a second reinforcing member to a second leaflet with fourth and fifth suture lines extending through the second leaflet and the second reinforcing member; forming a sixth suture line along the second reinforcing member; inserting a second commissure tab of the second leaflet assembly through the first commissure window; and forming a third securing loop that extends through the fifth and sixth suture lines and around a second axially extending window strut of the first commissure window; and tightening the third securing loop to fold the second reinforcing member and the second commissure tab around the second axially extending window strut.

Example 89. The method of any example herein, particularly example 88, further comprising forming a fourth securing loop that extends through the sixth suture line and the fourth suture line, such that the fourth securing loop extends around an end portion of the second leaflet assembly, and tightening the fourth securing loop to fold the end portion of the second leaflet assembly around the second axially extending window strut.

Example 90. The method of any example herein, particularly example 89, further comprising securing a wedge member to the first and second leaflet assemblies at a location radially outwards of the first commissure window.

Example 91. The method of any example herein, particularly examples 82-90, wherein no leaflet is sutured to any other leaflet or to itself.

Example 92. A prosthetic heart valve comprising: a radially expandable frame having a first end portion, a second end portion and a longitudinal axis extending between the first end portion and the second end portion, and a plurality of commissure windows; and a valvular structure disposed within and attached to the radially expandable frame, the valvular structure comprising a first leaflet having a first commissure tab and a first reinforcing member attached to the first commissure tab and a second leaflet having a second commissure tab and a second reinforcing member attached to the second commissure tab; wherein the first and second commissure tabs are positioned adjacent each other and extend through a first commissure window of the plurality of commissure windows, the first commissure window being defined by first and second axially extending struts, wherein the first reinforcing member is attached to the first commissure tab by a first suture line extending through the first leaflet and the first reinforcing member, and wherein the second reinforcing member is attached to the second commissure tab by a second suture line extending through the second leaflet and the second reinforcing member.

Example 93. The prosthetic heart valve of claim 92, wherein the first suture line and the second suture line are positioned radially outwards of the first commissure window.

Example 94. The prosthetic heart valve of either claim 92 or claim 93, wherein the first commissure tab and the first reinforcing member extend over a first face of the first axially extending strut on an interior of the commissure window and a second face of the first axially extending strut on an exterior of the frame, and wherein the second commissure tab and the second reinforcing member extend over a first face of the second axially extending strut on the interior of commissure window and a second face of the second axially extending strut on the exterior of the frame.

Example 95. The prosthetic heart valve of claim 94, wherein the first reinforcing member is positioned between the first and second faces of the first axially extending strut and the first commissure tab, and wherein the second reinforcing member is positioned between the first and second faces of the second axially extending strut and the second commissure tab.

Example 96. The prosthetic heart valve of either of claim 94 or claim 95, wherein the first suture line is adjacent to a first corner of the first axially extending strut at an interface between the first and second faces thereof, and wherein the second suture line is adjacent to a first corner of the second axially extending strut at an interface between the first and second faces thereof.

Example 97. The prosthetic heart valve of any of claims 92-96, wherein a wedge member is positioned between the first commissure tab and the second commissure tab at a location where the first and second commissure tabs exit the commissure window, and wherein one or more securing loops connect the wedge member to the first and second suture lines.

Example 98. The prosthetic heart valve of claim 97, wherein the one or more connecting loops comprise a first securing loop connecting the wedge member to the first suture line and a second securing loop connecting the wedge member to the second suture line.

Example 99. The prosthetic heart valve of claim 98, wherein each of the first suture line and the second suture line comprises a plurality of in-and-out stitches.

Example 100. The prosthetic heart valve of claim 99, wherein the first securing loop passes through at least a portion of the in-and-out stitches of the first suture line, and wherein the second securing loop passes through at least a portion of the in-and-out stitches of the second suture line.

Example 101. The prosthetic heart valve of any of claims 94-100, wherein the first reinforcing member extends over a third face of the first axially extending strut on an exterior of the commissure window and a fourth face of the first axially extending strut on an interior of the frame, and wherein the second reinforcing member extends over a third face of the first axially extending strut on the exterior of the commissure window and a fourth face of the first axially extending strut on the interior of the frame.

Example 102. The prosthetic heart valve of any of claims 92-101, wherein the first reinforcing member is wrapped around the first axially extending strut, and where the second reinforcing member is wrapped around the second axially extending strut.

Example 103. The prosthetic heart valve of any of claims 92-102, wherein the first reinforcing member comprises a third suture line extending through the first reinforcing member without extending through the first commissure tab, and where the second reinforcing member comprises a fourth suture line extending through the second reinforcing member without extending through the second commissure tab.

Example 104. The prosthetic heart valve of claim 103, wherein a portion of the first reinforcing member including the third suture line extends over and is exterior of the first commissure tab, and wherein a portion of the second reinforcing member including the fourth suture line extends over and is exterior of the second commissure tab.

Example 105. The prosthetic heart valve of claim 104, wherein the third suture line is connected to the fourth suture line via an outer securing loop.

Example 106. The prosthetic heart valve of claim 105, wherein each of the third suture line and the fourth suture line comprises a plurality of in-and-out stitches.

Example 107. The prosthetic heart valve of claim 106, wherein the outer securing loop passes through at least a portion of the in-and-out stitches of the third suture line and through at least a portion of the in-and-out stitches of the fourth suture line.

Example 108. The prosthetic heart valve of any of claims 92-102, wherein the first reinforcing member comprises first folded portion, and where the second reinforcing member comprises a second folded portion.

Example 109. The prosthetic heart valve of claim 108, wherein the first folded portion extends over and is exterior of the first commissure tab, and wherein the second folded portion extends over and is exterior of the second commissure tab.

Example 110. The prosthetic heart valve of claim 109, wherein the first folded portion is connected to the second folded portion via an outer securing loop.

Example 111. The prosthetic heart valve of any of claims 92-110, wherein the first leaflet is not sutured to the second leaflet.

Example 112. The prosthetic heart valve of any of claims 92-111, wherein the first commissure tab is not sutured to any portion of the first leaflet, and wherein the second commissure tab is not sutured to any portion of the second leaflet.

Example 113. The prosthetic heart valve of any of claims 92-112, wherein the first reinforcing member is attached to the first commissure tab with only the first suture line and the second reinforcing member is attached to the second commissure tab with only the second suture line.

Example 114. A method of assembling a prosthetic heart valve, comprising: assembling a first leaflet assembly by suturing a first reinforcing member to a commissure tab of a first leaflet with a first suture line extending through the first commissure tab and the first reinforcing member; assembling a second leaflet assembly by suturing a second reinforcing member to a second commissure tab of a second leaflet with a second suture line extending through the second commissure tab and the second reinforcing member; inserting the first and second commissure tabs through a commissure window of a frame of the prosthetic heart valve, the commissure window formed by a first axially extending strut and a second axially extending strut; extending the first commissure tab over at least an exterior face of the first axially extending strut, and extending the second commissure tab over at least an exterior face of the second axially extending strut; wrapping the first reinforcing member around a first axially extending strut of the commissure window, and wrapping the second reinforcing member around a second axially extending strut of the commissure window, wherein the first reinforcing member is disposed between the first axially extending strut and the first commissure tab, and wherein the second reinforcing member is disposed between the second axially extending strut and the second commissure tab; positioning a wedge member between the first and second commissure tabs at a location where they exit the commissure window; and forming a first securing loop that extends through the wedge member and the first suture line, and forming a second securing loop that extends through the wedge member and the second suture line.

Example 115. The method of claim 114, further comprising: wrapping a portion of the first reinforcing member including a third suture line over the first commissure tab, and wrapping a portion of the second reinforcing member including a fourth suture line over the second commissure tab, wherein the third suture line extends only through the first reinforcing member and the fourth suture line extends only through the second reinforcing member; and forming an exterior securing loop between the third suture line and the fourth suture line.

Example 116. The method of claim 114, further comprising: wrapping a first folded portion of the first reinforcing member over the first commissure tab, and wrapping a second folded portion of the second reinforcing member over the second commissure tab; and forming an exterior securing loop between the first folded portion and the second folded portion.

Example 117. A method comprising: sterilizing the commissure assembly, the radially expandable frame, the leaflets, or the prosthetic heart valve of any preceding claim.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A prosthetic heart valve, comprising:
a radially expandable frame having a first end portion, a second end portion, and a longitudinal axis extending between the first end portion and the second end portion, and a plurality of commissure windows; and
a valvular structure disposed within and attached to the radially expandable frame, the valvular structure comprising at least a first leaflet having a first commissure tab and a first reinforcing member attached to the first commissure tab and a second leaflet having a second commissure tab and a second reinforcing member attached to the second commissure tab;
wherein the first and second commissure tabs are positioned adjacent each other and extend through a first commissure window of the plurality of commissure windows, the first commissure window being defined by first and second axially extending posts,
wherein the first reinforcing member is attached to the first commissure tab by a first suture line; and
wherein no suture line attaches any leaflet to itself or to another leaflet.

2. The prosthetic heart valve of claim 1, further comprising a second suture line, and a third suture line, wherein the second suture line is spaced apart from the first suture line along a length of the first reinforcing member and the third suture line is spaced apart from the second suture line along the length of the first reinforcing member.

3. The prosthetic heart valve of claim 2, wherein the first suture line and the second suture line pass through the first reinforcing member and the first commissure tab.

4. The prosthetic heart valve of claim 2, wherein the third suture line passes only through the first reinforcing member.

5. The prosthetic heart valve of claim 2, wherein the first suture line and the second suture line are positioned radially outwards of the first commissure window and the third suture line is positioned radially inwards of the first commissure window.

6. The prosthetic heart valve of claim 2, further comprising a first securing loop extending from the second suture line to the third suture line.

7. The prosthetic heart valve of claim 6, wherein the first securing loop extends around the first axially extending post.

8. The prosthetic heart valve of claim 6, wherein the valvular structure further comprises a second securing loop extending from the first suture line to the third suture line.

9. The prosthetic heart valve of claim 8, wherein the second securing loop extends around the first commissure tab.

10. The prosthetic heart valve of claim 1, wherein the first reinforcing member separates the first leaflet from the first axially extending post and the second reinforcing member separates the second leaflet from the second axially extending post.

11. The prosthetic heart valve of claim 1, wherein the first reinforcing member extends completely around the first axially extending post and the second reinforcing member extends completely around the second axially extending post.

12. A prosthetic heart valve comprising:
a radially expandable frame having a first end portion, a second end portion and a longitudinal axis extending between the first end portion and the second end portion, and a plurality of commissure windows; and
a valvular structure disposed within and attached to the radially expandable frame, the valvular structure comprising a first leaflet having a first commissure tab and a first reinforcing member attached to the first commissure tab and a second leaflet having a second commissure tab and a second reinforcing member attached to the second commissure tab;
wherein the first and second commissure tabs are positioned adjacent each other and extend through a first commissure window of the plurality of commissure windows, the first commissure window being defined by first and second axially extending struts,
wherein the first reinforcing member is attached to the first commissure tab by a first suture line extending through the first leaflet and the first reinforcing member, and wherein the second reinforcing member is attached to the second commissure tab by a second suture line extending through the second leaflet and the second reinforcing member.

13. The prosthetic heart valve of claim 12, wherein the first suture line and the second suture line are positioned radially outwards of the first commissure window.

14. The prosthetic heart valve of claim 12, wherein the first commissure tab and the first reinforcing member extend over a first face of the first axially extending strut on an interior of the commissure window and a second face of the first axially extending strut on an exterior of the frame, and wherein the second commissure tab and the second reinforcing member extend over a first face of the second axially extending strut on the interior of commissure window and a second face of the second axially extending strut on the exterior of the frame.

15. The prosthetic heart valve of claim 14, wherein the first reinforcing member is positioned between the first and second faces of the first axially extending strut and the first commissure tab, and wherein the second reinforcing member is positioned between the first and second faces of the second axially extending strut and the second commissure tab.

16. The prosthetic heart valve of claim 12, wherein a wedge member is positioned between the first commissure tab and the second commissure tab at a location where the first and second commissure tabs exit the commissure window, and wherein one or more securing loops connect the wedge member to the first and second suture lines.

17. The prosthetic heart valve of claim 16, wherein the one or more connecting loops comprise a first securing loop connecting the wedge member to the first suture line and a second securing loop connecting the wedge member to the second suture line.

18. The prosthetic heart valve of claim 12, wherein the first reinforcing member is wrapped around the first axially extending strut, and where the second reinforcing member is wrapped around the second axially extending strut.

19. The prosthetic heart valve of claim 12, wherein the first commissure tab is not sutured to any portion of the first leaflet, and wherein the second commissure tab is not sutured to any portion of the second leaflet.

20. A method of assembling a prosthetic heart valve, comprising:
assembling a first leaflet assembly by suturing a first reinforcing member to a commissure tab of a first leaflet with a first suture line extending through the first commissure tab and the first reinforcing member;
assembling a second leaflet assembly by suturing a second reinforcing member to a second commissure tab of a second leaflet with a second suture line extending through the second commissure tab and the second reinforcing member;
inserting the first and second commissure tabs through a commissure window of a frame of the prosthetic heart valve, the commissure window formed by a first axially extending strut and a second axially extending strut;
extending the first commissure tab over at least an exterior face of the first axially extending strut, and extending the second commissure tab over at least an exterior face of the second axially extending strut;
wrapping the first reinforcing member around a first axially extending strut of the commissure window, and wrapping the second reinforcing member around a second axially extending strut of the commissure window, wherein the first reinforcing member is disposed between the first axially extending strut and the first commissure tab, and wherein the second reinforcing member is disposed between the second axially extending strut and the second commissure tab;
positioning a wedge member between the first and second commissure tabs at a location where they exit the commissure window; and
forming a first securing loop that extends through the wedge member and the first suture line, and forming a second securing loop that extends through the wedge member and the second suture line.
